(19) United States
(12) Patent Application Publication
(10) Pub. No.: US 2013/0251725 A1
(43) Pub. Date: Sep. 26, 2013

(54) Anti-P-Selectin Antibody Formulation

(71) Applicant: HOFFMANN-LA ROCHE INC., Nutley, NJ (US)

(72) Inventors: Ulla Grauschopf, Riehen (CH); Michael Adler, Riehen (CH); Hanns-Christian Mahler, Basel (CH)

(73) Assignee: HOFFMANN-LA ROCHE INC., Nutley, NJ (US)

(21) Appl. No.: 13/788,204

(22) Filed: Mar. 7, 2013

(30) Foreign Application Priority Data
Mar. 8, 2012 (EP) 12158626.7

(51) Int. Cl. A61K 39/395

(52) U.S. Cl. CPC A61K 39/3955
USPC 424/142.1

(57) ABSTRACT

The present invention relates to a stable liquid pharmaceutical formulation comprising 40 mg/ml to 200 mg/ml of an antibody against P-selectin; 0.01% to 0.1% of a poloxamer; 5 mM to 100 mM of a buffer; and 100 mM to 500 mM of at least one stabilizer; at a pH in the range from 4.5 to 7.0.

### CROSS REFERENCE TO RELATED APPLICATIONS

This application claims the benefit of priority under 35 USC §119(a) to European patent application number 12158626.7, filed 8 Mar. 2012, the disclosure of which is incorporated herein by reference in its entirety.

### SEQUENCE LISTING

This application contains a Sequence Listing which has been submitted via EFS-Web and is hereby incorporated by reference in its entirety. Said ASCII copy, created on Mar. 6, 2013, is named P4878US_Sequence_Listing.TXT and is 29,196 bytes in size.

### FIELD OF THE INVENTION

The present invention relates to a stable pharmaceutical liquid formulation of an antibody molecule against P-selectin, a process for the preparation of said formulation and uses of the formulation.

### BACKGROUND

Antibodies against P-selectin are of therapeutic interest, in particular as medicaments for the treatment and prophylaxis of inflammatory and thrombotic disorders and cardiovascular diseases. Antibodies against P-selectin (CD62P, GMP-140, PADGEM, LECAM3) are for example described in WO 2005/100402. These antibodies inhibit the adhesion of leukocyte-like HL60 cells to purified P-selectin immobilized on microtiter plates in an adhesion assay with an IC₅₀ value of 0.08 to 0.5 μg/ml.

Low concentration formulations (15 mg/ml antibody) of anti-P-selectin antibodies in liquid form, lyophilized form or in liquid form reconstituted from a lyophilized form are disclosed in WO 2010/031720.

Antibody molecules, as part of the group of protein pharmaceuticals, are very susceptible to physical and chemical degradation. Chemical degradation includes any process that involves modification of the protein via bond formation or cleavage, yielding a new chemical entity. A variety of chemical reactions is known to affect proteins. These reactions can involve hydrolysis including cleavage of peptide bonds as well as deamidation, isomerization, oxidation and decomposition. Physical degradation refers to changes in the higher order structure and includes denaturation, adsorption to surfaces, aggregation and precipitation. Protein stability is influenced by the characteristics of the protein itself, e.g. the amino acid sequence, the glycosylation pattern, and by external influences, such as temperature, solvent pH, excipients, interfaces, or shear rates. So, it is important to define the optimal formulation conditions to protect the protein against degradation reactions during manufacturing, storage and administration. (Manning, M. C., et al. (1989), “Stability of protein pharmaceuticals”, Pharm Res 6(11), 903-918; Zheng, J. Y., Janis, L. J. (2005), “Influence of pH, buffer species, and storage temperature on physicochemical stability of a humanized monoclonal antibody LA298”, Int. J. Pharmaceutics 308, 46-51). Stable liquid formulations of therapeutic antibodies are particularly difficult to obtain when the formulation should include antibodies in a high concentration.

It is therefore an object of the present invention to provide a highly concentrated, stable formulation of an anti-P-selectin antibody with as few as necessary excipients, which enables the desired dosing and allows convenient administration of the antibody to a patient.

The formulation of the present invention shows good stability upon storage for 18 months at the intended storage temperature of 2 to 8° C. without formation of visible particles that will allow i.v. administration without the need of an in-line filter allowing greater administration convenience. Shaking and multiple freezing-thawing steps were applied to the liquid formulation to simulate physical stress conditions that potentially occur during manufacturing or transportation of the drug product. The formulation of the present invention shows good stability after applying shaking and freeze-thaw stress.

### SUMMARY

In one aspect, the invention refers to a stable liquid pharmaceutical formulation comprising:
40 mg/ml to 200 mg/ml of an antibody against P-selectin;
0.01% to 0.1% of a poloxamer;
5 mM to 100 mM of a buffer;
100 mM to 500 mM of at least one stabilizer;

at a pH in the range from 4.5 to 7.0.

In one embodiment, the concentration of the antibody against P-selectin is in the range of 40 mg/ml to 100 mg/ml, particularly of 50 mg/ml.

In another embodiment, the invention relates to the pharmaceutical formulation, wherein the poloxamer is Poloxamer 188.

In a further embodiment, the poloxamer is present in a concentration in the range from 0.01% to 0.05%, particularly of 0.02%.

In one embodiment, the invention relates to the pharmaceutical formulation, wherein the buffer is a histidine buffer, particularly a histidine acetate buffer.

In a further embodiment, the buffer has a concentration in the range of 10 to 30 mM, particularly of 20 mM.

In another embodiment, the pH of the formulation is in the range of 5.0 to 6.0, particularly at 5.5.

In one embodiment, the invention is concerned with the pharmaceutical formulation, wherein at least one stabilizer is selected from the group consisting of sugars, polyols and amino acids. More particularly, the stable liquid formulation of the invention comprises one stabilizer selected from the group consisting of sugars, polyols and amino acids.

In a further embodiment, the at least one stabilizer is present in a concentration in the range from 140 to 250 mM, particularly in the range from 210 to 230 mM.

In another embodiment, the stabilizer is selected from the group consisting of trehalose, sucrose, sorbitol and arginine hydrochloride.

In one embodiment, the invention refers to the pharmaceutical formulation, wherein the antibody against P-selectin is a human or humanized antibody.

In a further embodiment, the antibody against P-selectin comprises a variable region independently selected from the group consisting of
a) the heavy chain variable domain defined by amino acid sequence SEQ ID NO:2 and the light chain variable domain defined by SEQ ID NO:1;
b) the heavy chain variable domain defined by amino acid sequence SEQ ID NO:4 and the light chain variable domain defined by SEQ ID NO:3;
c) the heavy chain variable domain defined by amino acid sequence SEQ ID NO:6 and the light chain variable domain defined by SEQ ID NO:5;
d) the heavy chain variable domain defined by amino acid sequence SEQ ID NO:8 and the light chain variable domain defined by SEQ ID NO:7;
e) the heavy chain variable domain defined by amino acid sequence SEQ ID NO:10 and the light chain variable domain defined by SEQ ID NO:9;
f) the heavy chain variable domain defined by amino acid sequence SEQ ID NO:12 and the light chain variable domain defined by SEQ ID NO:11;
g) the heavy chain variable domain defined by amino acid sequence SEQ ID NO:14 and the light chain variable domain defined by SEQ ID NO:13;
h) the heavy chain variable domain defined by amino acid sequence SEQ ID NO:16 and the light chain variable domain defined by SEQ ID NO:15;
i) the heavy chain variable domain defined by amino acid sequence SEQ ID NO:18 and the light chain variable domain defined by SEQ ID NO:17;
j) the heavy chain variable domain defined by amino acid sequence SEQ ID NO:20 and the light chain variable domain defined by SEQ ID NO:19; and
k) the heavy chain variable domain defined by amino acid sequence SEQ ID NO:22 and the light chain variable domain defined by SEQ ID NO:21.

In a further embodiment, the invention relates to the pharmaceutical formulation, wherein the heavy chain variable domain of the antibody against P-selectin comprises the amino acid sequence SEQ ID NO:4 and the light chain variable domain of the antibody against P-comprises SEQ ID NO:3.

In another embodiment, the invention is concerned with the pharmaceutical formulation, wherein the antibody against P-selectin is a human antibody comprising the heavy chain of SEQ ID NO:24 and the light chain of SEQ ID NO:23.

Another aspect of the invention provides a stable liquid pharmaceutical formulation comprising:

50 mg/ml of a human antibody against P-selectin comprising the heavy chain of SEQ ID NO:24 and the light chain of SEQ ID NO:23; and
(i) 0.02% Poloxamer 188,
   230 mM trehalose, and
   20 mM histidine acetate buffer at pH 5.5; or
(ii) 0.02% Poloxamer 188,
210 mM sucrose, and
   20 mM histidine acetate buffer at pH 5.5; or
(iii) 0.02% Poloxamer 188,
   145 mM arginine hydrochloride, and
   20 mM histidine acetate buffer at pH 5.5; or
(iv) 0.02% Poloxamer 188,
   230 mM sorbitol, and
   20 mM histidine acetate buffer at pH 5.5.

In yet another embodiment, the invention relates to the stable liquid pharmaceutical formulation, wherein methionine is present as a second stabilizer, particularly in a concentration of 5 to 25 mM.

For example, such formulation may comprise:

50 mg/ml of a human antibody against P-selectin comprising the heavy chain of SEQ ID NO:24 and the light chain of SEQ ID NO:23; and
(v) 0.02% Poloxamer 188,
   230 mM trehalose,
   10 mM methionine, and
   20 mM histidine acetate buffer at pH 5.5; or
(vi) 0.02% Poloxamer 188,
   210 mM sucrose,
   10 mM methionine, and
   20 mM histidine acetate buffer at pH 5.5; or
(vii) 0.02% Poloxamer 188,
   145 mM arginine hydrochloride,
   10 mM methionine, and
   20 mM histidine acetate buffer at pH 5.5; or
(viii) 0.02% Poloxamer 188,
   230 mM sorbitol,
   10 mM methionine, and
   20 mM histidine acetate buffer at pH 5.5.

In a further aspect, the invention provides a stable liquid pharmaceutical formulation comprising:
(a) 40 mg/ml of a human antibody against P-selectin comprising the heavy chain of SEQ ID NO:24 and the light chain of SEQ ID NO:23,
   0.01% Poloxamer 188,
   210 mM sucrose, and
   20 mM histidine acetate buffer at pH 5.0; or
(b) 60 mg/ml of a human antibody against P-selectin comprising the heavy chain of SEQ ID NO:24 and the light chain of SEQ ID NO:23,
   0.01% Poloxamer 188,
   210 mM sucrose, and
   20 mM histidine acetate buffer at pH 5.0; or
(c) 40 mg/ml of a human antibody against P-selectin comprising the heavy chain of SEQ ID NO:24 and the light chain of SEQ ID NO:23,
   0.03% Poloxamer 188,
   210 mM sucrose, and
   20 mM histidine acetate buffer at pH 5.0; or
(d) 60 mg/ml of a human antibody against P-selectin comprising the heavy chain of SEQ ID NO:24 and the light chain of SEQ ID NO:23,
   0.03% Poloxamer 188,
   210 mM sucrose, and
   20 mM histidine acetate buffer at pH 5.0; or
(e) 40 mg/ml of a human antibody against P-selectin comprising the heavy chain of SEQ ID NO:24 and the light chain of SEQ ID NO:23,
   0.01% Poloxamer 188,
   210 mM sucrose, and
   20 mM histidine acetate buffer at pH 6.0; or
(f) 60 mg/ml of a human antibody against P-selectin comprising the heavy chain of SEQ ID NO:24 and the light chain of SEQ ID NO:23,
   0.01% Poloxamer 188,
   210 mM sucrose, and
   20 mM histidine acetate buffer at pH 6.0; or
(g) 40 mg/ml of a human antibody against P-selectin comprising the heavy chain of SEQ ID NO:24 and the light chain of SEQ ID NO:23,
   0.03% Poloxamer 188,
   210 mM sucrose, and
   20 mM histidine acetate buffer at pH 6.0; or
(h) 60 mg/ml of a human antibody against P-selectin comprising the heavy chain of SEQ ID NO:24 and the light chain of SEQ ID NO:23,
   0.03% Poloxamer 188,
   210 mM sucrose, and
   20 mM histidine acetate buffer at pH 6.0; or
(i) 50 mg/ml of a human antibody against P-selectin comprising the heavy chain of SEQ ID NO:24 and the light chain of SEQ ID NO:23,
   0.02% Poloxamer 188,
   210 mM sucrose, and
   20 mM histidine acetate buffer at pH 5.5.

In another aspect of the invention is provided the stable liquid pharmaceutical formulation for use in the prevention or treatment of inflammatory and thrombotic diseases as well as vascular disease pathologies driven by inflammatory and/or thrombotic processes. Such diseases include vascular diseases with underlying atherothrombosis and vascular narrowing such as coronary artery disease (CAD), acute coronary syndrome (ACS), peripheral arterial disease (PAD), peripheral arterial occlusive disease (PAOD), critical limb ischemia (CLI), stenosis after coronary artery bypass grafting (CABG), coronary vein graft disease, restenosis after percutaneous coronary intervention (PCI), dialysis shunt stenosis, dialysis shunt occlusion as well as arterial and deep venous thrombosis and thrombotic thrombocytopenic purpura (TPP). Other applications are the prevention and treatment of post-ischemic tissue damage caused by myocardial infarction, cerebral ischemic event (e.g. stroke), renal infarction, acute kidney injury, organ transplant rejection and acute leukocyte-mediated lung-injury. The stable liquid pharmaceutical formulation of the antibody against P-selectin is also suitable for the treatment of septic shock, allergic conditions, asthma, chronic obstructive pulmonary disease (COPD), transplant vasculopathy, acute pancreatitis, inflammatory bowel disease, rheumatoid arthritis, atopic dermatitis, psoriasis, sickle cell disease and prevention of tumor metastasis.

More particularly, the stable liquid pharmaceutical formulation of the antibody against P-selectin is for use in the prevention or treatment of acute coronary syndrome (ACS), stenosis after coronary artery bypass grafting (CABG), coronary vein graft disease, restenosis after percutaneous coronary intervention (PCI) such as angioplasty or stent placement, peripheral arterial disease (PAD), peripheral arterial occlusive disease (PAOD) and critical limb ischemia (CLI).

Another aspect of the invention is the use of the stable liquid pharmaceutical formulation for the preparation of a medicament for use in the prevention or treatment of inflammatory and thrombotic diseases as well as vascular disease pathologies driven by inflammatory and/or thrombotic processes. Such diseases include vascular diseases with underlying atherothrombosis and vascular narrowing such as coronary artery disease (CAD), acute coronary syndrome (ACS), peripheral arterial disease (PAD), peripheral arterial occlusive disease (PAOD), critical limb ischemia (CLI), stenosis after coronary artery bypass grafting (CABG), coronary vein graft disease, restenosis after percutaneous coronary intervention (PCI), dialysis shunt stenosis, dialysis shunt occlusion as well as arterial and deep venous thrombosis and thrombotic thrombocytopenic purpara (TPP), the prevention and treatment of post-ischemic tissue damage caused by myocardial infarction, cerebral ischemic event (e.g. stroke), renal infarction, acute kidney injury, organ transplant rejection and acute leukocyte-mediated lung-injury, the treatment of septic shock, allergic conditions, asthma, chronic obstructive pulmonary disease (COPD), transplant vasculopathy, acute pancreatitis, inflammatory bowel disease, rheumatoid arthritis, atopic dermatitis, psoriasis, sickle cell disease and prevention of tumor metastasis.

More particularly, the invention is concerned with the use of the stable liquid pharmaceutical formulation of the antibody against P-selectin for the preparation of a medicament for use in the prevention or treatment of acute coronary syndrome (ACS), stenosis after coronary artery bypass grafting (CABG), coronary vein graft disease, restenosis after percutaneous coronary intervention (PCI) such as angioplasty or stent placement, peripheral arterial disease (PAD), peripheral arterial occlusive disease (PAOD) and critical limb ischemia (CLI).

Another aspect of the invention is a method for the prevention or treatment of inflammatory and thrombotic diseases as well as vascular disease pathologies driven by inflammatory and/or thrombotic processes, particularly for the prevention or treatment of vascular diseases with underlying atherothrombosis and vascular narrowing such as coronary artery disease (CAD), acute coronary syndrome (ACS), peripheral arterial disease (PAD), peripheral arterial occlusive disease (PAOD), critical limb ischemia (CLI), stenosis after coronary artery bypass grafting (CABG), coronary vein graft disease, restenosis after percutaneous coronary intervention (PCI), dialysis shunt stenosis, dialysis shunt occlusion as well as arterial and deep venous thrombosis and thrombotic thrombocytopenic purpura (TPP), the prevention and treatment of post-ischemic tissue damage caused by myocardial infarction, cerebral ischemic event (e.g. stroke), renal infarction, acute kidney injury, organ transplant rejection and acute leukocyte-mediated lung-injury, the treatment of septic shock, allergic conditions, asthma, chronic obstructive pulmonary disease (COPD), transplant vasculopathy, acute pancreatitis, inflammatory bowel disease, rheumatoid arthritis, atopic dermatitis, psoriasis, sickle cell disease and prevention of tumor metastasis, which method comprises administering the stable liquid pharmaceutical formulation according to the invention.

### DETAILED DESCRIPTION OF THE INVENTION

The present invention relates to a stable pharmaceutical liquid formulation comprising an antibody against P-selectin in high concentration.

The term “pharmaceutical formulation” refers to preparations which are in such form as to permit the biological activity of the active ingredients to be unequivocally effective, and which contain no additional components which are toxic to the subjects to which the formulation is administered.

The term “liquid” as used herein in connection with the formulation according to the invention denotes a formulation which is liquid at a temperature of at least about 2° C. to about 8° C. under atmospheric pressure.

A “stable” formulation is one in which the protein therein, e.g. the antibody, essentially retains its physical and chemical stability and thus its biological activity upon storage.

A “stable liquid pharmaceutical antibody formulation” is a liquid antibody formulation with no significant changes observed at a refrigerated temperature (2-8° C.) for at least 12 months, particularly 2 years, and more particularly 3 years. The criteria for stability are the following: no more than 10%, particularly 5%, of antibody monomer is degraded as measured by size exclusion chromatography (SEC-HPLC). Furthermore, the solution is colorless or clear to slightly opalescent by visual analysis. The protein concentration of the formulation has no more than +/−10% change. No more than 10%, particularly 5% of aggregation is formed. The stability is measured by methods known in the art such UV spectroscopy, size exclusion chromatography (SEC-HPLC), Ion-Exchange Chromatography (IE-HPLC), turbidimetry and visual inspection.

The term “P-selectin” refers to a 140 kDa protein expressed by human platelets and endothelial cells, as described by Hsu-Lin et al., J Biol Chem 259: 9121 (1984), and Mc Ever et al., J Clin Invest 84:92 (1989). This type I transmembrane glycoprotein (SwissProt sequence P16109) is composed of an NH₂-terminal lectin domain, followed by an epidermal growth factor (EGF)-like domain and nine consensus repeat domains. It is anchored in the membrane by a single transmembrane domain and contains a small cytoplasmic tail.

The terms “antibody against P-selectin” and “anti-P-selectin antibody” refer to an antibody that is capable of binding to P-selectin with sufficient affinity such that the antibody is useful as a diagnostic and/or therapeutic agent in targeting P-selectin. The term “binding to P-selectin” as used herein means the binding of the antibody to P-selectin in either a BIAcore™ assay (Pharmacia Biosensor AB, Uppsala, Sweden) or in an ELISA in which either purified P-selectin or P-selectin CHO transfectants are coated onto microtiter plates.

In the BIAcore assay the antibody is bound to a surface and binding of P-selectin is measured by Surface Plasmon Resonance (SPR). The affinity of the binding is defined by the terms ka (rate constant for the association of the antibody from the antibody/antigen complex), kd (dissociation constant), and K_{D} (kd/ka). The antibodies that are particularly useful for the invention show a K_{D} of 10⁻⁸ or less, particularly of about 10⁻¹¹ to 10⁻⁹ M.

In the P-selectin-specific ELISA purified P-selectin expressing CHO transfectants are coated onto microtiter plates and the binding of the antibody to P-selectin is detected with a biotinylated anti-human IgG and the usual steps of an ELISA. The EC₅₀ values in this assay of the antibodies that are particularly useful for the invention range between 0.01 and 0.08 μg/ml, more particularly between 0.01 and 0.04 μg/ml.

The term “antibody” encompasses the various forms of antibody structures including but not being limited to whole antibodies and antibody fragments. The antibody according to the invention is in particular a human antibody, a humanized antibody, chimeric antibody, antibody fragment, or further genetically engineered antibody as long as the characteristic properties according to the invention are retained. More particularly, the antibody is a human or humanized monoclonal antibody, especially a recombinant human antibody.

“Antibody fragments” comprise a portion of a full length antibody, preferably the variable domain thereof, or at least the antigen binding site thereof. Examples of antibody fragments indude diabodies, single-chain antibody molecules, and multispecific antibodies formed from antibody fragments. scFv antibodies are, e.g. described in Houston, J. S., Methods in Enzymol. 203 (1991) 46-96). In addition, antibody fragments comprise single chain polypeptides having the characteristics of a V_{H} domain, namely being able to assemble together with a V_{L} domain, or of a V_{L} domain binding to Aβ, namely being able to assemble together with a V_{H} domain to a functional antigen binding site and thereby providing the property.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are of particular interest. Other forms of “chimeric antibodies” encompassed by the present invention are those in which the constant region has been modified or changed from that of the original antibody to generate the properties according to the invention, especially in regard to Clq binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to Clq binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to Clq binding and/or FcR binding, e.g. by “class switching” i.e. change or mutation of Fc parts (e.g. from IgG1 to IgG4 and/or IgG1/IgG4 mutation.).

The human antibodies against P-selectin as used herein are characterized by a high selectivity for P-selectin vs. E- and L-selectin. Such antibodies according to the invention bind to P-selectin expressing cells with EC₅₀ values in the range of 0.01 and 0.07 μg/ml. EC₅₀ values on Eselectin and L-selectin expressing cells are preferably above 100 μg/ml.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable region” (variable region of a light chain (V_{L}), variable region of a heavy chain (V_{H})) or “variable domain” as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention. The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The antibodies that are particularly useful for the invention are particularly capable of binding to P-selectin in the presence of the P-selectin fragment aa 60-75 (SwissProt sequence P16109) and/or do not competitively inhibit the binding of an antibody secreted by a cell line designated ATCC® Accession No. HB11041 to P-selectin.

The “constant regions” or “constant domains” are not involved directly in binding an antibody to an antigen, but exhibit various effector functions. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3 and IgG4, IgA1 and IgA2. The antibodies used in the invention are particularly of IgG type, more particularly of IgG1 or IgG4 human subtype.

An antibody that is particularly useful for the invention is characterized in that it contains an Fc part derived from human origin, and in that it does not bind to complement factor Clq and not to Fcγ receptors on effector cells. In particular, the antibody is characterized in that it is an antibody of human subclass IgG1, containing at least one mutation in L234, L235, D270, N297, E318, K320, K322, P331 and/or P329 or an antibody of human subclass IgG4, containing at least one mutation in L235 and S228 (numbering according to EU index). More particularly, it is an antibody of human subclass IgG4 wherein S228 is replaced by P and L235 is replaced by E (SPLE mutation). Such an antibody is defined as IgG4v1 (S228P; L235E). Further antibodies of particular interest are those defined as IgG1v1 (PVA-236; GLPSS331 as specified by E233P; L234V; L235A; delta G236; A327G; A330S; P331S) and IgG1v2 (L234A; L235A).

The concentration of the antibody against P-selectin comprised in the pharmaceutical formulation is in the range of 40 mg/ml to 200 mg/ml, particularly in the range of 40 mg/ml to 100 mg/ml, more particularly in the range of 40 mg/ml to 60 mg/ml and most particularly of 50 mg/ml.

The pharmaceutical formulation of the present invention comprises a poloxamer as surfactant to reduce aggregation of the antibodies and particle formation. The term “poloxamer” as used herein includes a polyoxyethylene-polyoxypropylene triblock copolymer composed of a central hydrophobic chain of polyoxypropylene flanked by two hydrophilic chains of polyoxyethylene known as poloxamer 188, sold under the trade name PLURONIC® F68 by BASF (Parsippany, N.J.). Other poloxamers which may be utilized in the formulations of the present invention include poloxamer 403 (sold as PLURONIC® P123), poloxamer 407 (sold as PLURONIC® P127), poloxamer 402 (sold as PLURONIC® P122), poloxamer 181 (sold as PLURONIC® L61), poloxamer 401 (sold as PLURONIC® L121), poloxamer 185 (sold as PLURONIC® P65), and poloxamer 338 (sold as PLURONIC® F108).

The term “surfactant” as used herein denotes a pharmaceutically acceptable excipient which is used to protect protein formulations against mechanical stresses like agitation and shearing. Examples of pharmaceutically acceptable surfactants include polyoxyethylensorbitan fatty acid esters (Tween), polyoxyethylene alkyl ethers (for example those sold under the trademark Brij™) and polyoxyethylene-polyoxypropylene copolymer (Poloxamer, Pluronic). Examples of polyoxyethylenesorbitan-fatty acid esters are polysorbate 20 (sold under the trademark Tween® 20) and polysorbate 80 (sold under the trademark Tween® 80).

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers, acetate-buffers, arginine-buffers, phosphate-buffers or mixtures thereof. Buffers of particular interest comprise L-histidine or mixtures of L-histidine and L-histidine hydrochloride with pH adjustment with an acid or a base known in the art. The abovementioned buffers are generally used in an amount of about 5 mM to about 100 mM, particularly of about 10 mM to about 30 mM and more particularly of about 20 mM. Independently from the buffer used, the pH can be adjusted to a value in the range from 4.5 to 7.0 and particularly to a value in the range from 5.0 to 6.0 and most particularly to pH 5.5±0.03 with an acid or a base known in the art, e.g. hydrochloric acid, acetic acid, phosphoric acid, sulfuric acid and citric acid, sodium hydroxide and potassium hydroxide.

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit Rev Ther Drug Carrier Syst 10(4):307-77, Wang (1999) Int J Pharm 185(2):129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-β-cyclodextrine, sulfobutylethyl-β-cyclodextrin, β-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. Stabilizers that are particularly used in the present invention, are selected from the group consisting of sugars, polyols and amino acids. More particularly, the stabilizers are selected from the group consisting of sucrose, trehalose, sorbitol and arginine hydrochloride. Stabilizers can be present in the formulation in an amount of about 100 mM to about 500 mM, particularly in an amount of about 140 to about 250 mM and more particularly in an amount of about 210 mM to about 230 mM. More particularly, sucrose or trehalose are used as stabilizers in an amount of about 210 mM to about 230 mM.

In some embodiments, the stable liquid pharmaceutical formulation of the present invention comprises an antioxidant as a second stabilizer. An “antioxidant” is a pharmaceutically acceptable excipient, which prevents oxidation of the active pharmaceutical ingredient. Antioxidants include but are not limited to chelating agents such as EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol, benzyl alcohol and n-acetyl cysteine. Antioxidants can be used in an amount of about 0.01 to about 100 mM, particularly in an amount of about 5 to about 50 mM and more particularly in an amount of about 5 to about 25 mM. In particular, methionine is chosen as a second stabilizer, particularly in a concentration of about 5 to about 25 mM, more particularly in a concentration of about 10 mM.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides, the monosaccharides and oligosaccharides are water soluble. Examples of oligosaccharides include sucrose, trehalose, lactose, maltose and raffinose. In particular, sugars are selected from sucrose and trehalose.

The term “amino acid” as used herein denotes a pharmaceutically acceptable organic molecule possessing an amino moiety located at α-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Amino acids are generally used in an amount of about 5 to 500 mM, particularly in an amount of about 5 to about 200 mM and more particularly in an amount of about 100 to about 150 mM.

The term “polyols” as used herein denotes pharmaceutically acceptable alcohols with more than one hydroxy group. Suitable polyols comprise to but are not limited to mannitol, sorbitol, glycerine, dextran, glycerol, arabitol, propylene glycol, polyethylene glycol, and combinations thereof. Polyols can be used in an amount of about 10 mM to about 500 mM, particularly in an amount of about 10 to about 250 mM and more particularly in an amount of about 200 to about 250 mM.

The term “stabilizers” also includes lyoprotectants. The term “lyoprotectant” denotes a pharmaceutical acceptable excipient, which protects the labile active ingredient (e.g. a protein) against destabilizing conditions during the lyophilisation process, subsequent storage and reconstitution. Lyoprotectants comprise but are not limited to the group consisting of sugars, polyols (such as e.g. sugar alcohols) and amino acids. In particular, lyoprotectants can be selected from the group consisting of sugars such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine or mixtures thereof. Lyoprotectants are generally used in an amount of about 10 to about 500 mM, particularly in an amount of about 10 to about 250 mM and more particularly in an amount of about 100 to about 250 mM.

The pharmaceutical formulation may also contain tonicity agents. The term “tonicity agents” as used herein denotes pharmaceutically acceptable tonicity agents which are used to modulate the tonicity of the formulation. The formulation can be hypotonic, isotonic or hypertonic. Isotonicity in general relates to the osmostic pressure relative of a solution usually relative to that of human blood serum. The formulation according to the invention can be hypotonic, isotonic or hypertonic but will preferably be isotonic. An isotonic formulation is liquid or liquid reconstituted from a solid form, e.g. from a lyophilised form and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids, sugars, in particular glucose. Tonicity agents are generally used in an amount of about 5 mM to about 500 mM. Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is trehalose.

The pharmaceutical formulation may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

The stable liquid pharmaceutical formulation of the antibody against P-selectin according to the invention can be used in the prevention or treatment of thrombotic disorders and inflammatory diseases, particularly in the prevention or treatment of disorders or diseases selected from the group consisting of vascular disorders such as vascular narrowing, atherosclerosis, arterial and deep venous thrombosis, acute coronary syndrome (ACS), coronary artery disease (CAD), coronary heart disease, prevention of subsequent cardiovascular events in patients treated with coronary artery bypass graft (CABG) surgery or percutaneous coronary intervention (PCI), stenosis, restenosis after angioplasty or stent placement, peripheral arterial disease (PAD), peripheral arterial occlusive disease (PAOD), critical limb ischemia (CLI), post-ischemic leukocyte-mediated tissue damage caused by myocardial infarction, cerebral ischemic event (e.g. stroke), hemodialysis shunt stenosis, dialysis shunt occlusion, renal infarction, acute kidney injury, sepsis, acute leukocyte-mediated lung-injury, allergic reactions such as asthma, transplant vasculopathy, prevention of organ transplant rejection, acute pancreatitis, inflammatory bowel disease, autoimmune diseases such as rheumatoid arthritis, thrombotic thrombocytopenic purpura (TPP), psoriasis, sickle cell disease and prevention of tumor metastasis by inhibiting the adhesion of circulating cancer cells.

More particularly, the stable liquid pharmaceutical formulation of the antibody against P-selectin can be used in the prevention or treatment of acute coronary syndrome (ACS), subsequent cardiovascular events in patients treated with coronary artery bypass graft (CABG) surgery or percutaneous coronary intervention (PCI), stenosis, restenosis after angioplasty or stent placement, peripheral arterial disease (PAD), peripheral arterial occlusive disease (PAOD) and critical limb ischemia (CLI).

The stable liquid pharmaceutical formulation according to the invention can be administered by intravenous (i.v.), subcutaneous (s.c.) or any other parental administration means such as those known in the pharmaceutical art.

In view of their high stability the pharmaceutical formulation according to the invention can be administered i.v. without the need of an in-line filter and is thus much more convenient to handle than conventional formulations that need to be administered with an in-line filter. In-line filters such as Sterifix® have to be installed in the infusion line of i.v. medications to prevent the administration of any particles, air, or microorganisms that may be in the i.v. solution or line. Particles of 5 to 20 microns size and larger have the capability of obstructing blood flow through pulmonary capillaries, which could lead to complications such as pulmonary embolism. Foreign particles can also cause phlebitis at the injection site and filters may help to reduce the incidence of phlebitis.

The stable formulations to be used for in vivo administration must be sterile. This is readily accomplished by filtration through sterile filtration membranes.

The stable liquid pharmaceutical formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found herein after.

The stable liquid pharmaceutical formulations according to the invention can also be in a lyophilized form or in a liquid form reconstituted from the lyophilized form. The “lyophilized form” is manufactured by freeze-drying methods known in the art. The lyophilizate usually has a residual moisture content of about 0.1 to 5% (w/w) and is present as a powder or a physically stable cake. The “reconstituted form” can be obtained from the lyophilizate by a fast dissolution after addition of reconstitution medium. Suitable reconstitution media comprise but are not limited to water for injection (WFI), bacteriostatic water for injection (BWFI), sodium chloride solutions (e.g. 0.9% (w/v) NaCl), glucose solutions (e.g. 5% (w/v) glucose), surfactant-containing solutions (e.g. 0.01% (w/v) polysorbate 20 and pH-buffered solutions (e.g. phosphate-buffered solutions).

Production of the Antibodies

The antibodies against P-selectin that are particularly useful for the invention can be produced from hybridoma cell lines, in particular from the hybridoma cell lines hu-Mab<P-selectin>LC 1004-001 (antibody HuMab 001), hu-Mab<P-selectin>LC 1004-002 (antibody HuMab 002) and hu-Mab<P-selectin>LC 1004-017 (antibody HuMab 017), that were deposited on 30 Mar. 2004 under the Budapest Treaty on the international recognition of the deposit of microorganisms for the purposes of patent procedure, with the Deutsche Sammlung von Mikroorganismen and Zellkulturen GmbH (DSMZ), Germany and obtained the Deposition Nos. DSM ACC2640, DSM ACC2641 and DSM ACC2642, respectively.

In particular, antibodies that are particularly useful for the invention are produced by recombinant means. Such methods are widely known in the state of the art and described, for example, in the review articles of Makrides, S. C., Protein Expr. Purif 17 (1999) 183-202; Geisse, S., et al., Protein Expr. Purif 8 (1996) 271-282; Kaufman, R. J., Mol. Biotechnol. 16 (2000) 151-161; or Werner, R. G., Drug Res. 48 (1998) 870-880. The methods comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. DNA and RNA encoding the antibodies are readily isolated and sequenced using conventional procedures. The hybridoma cells can serve as a source of such DNA and RNA. Such nucleic acid may be readily isolated and sequenced using conventional procedures (e.g., by using oligonucleotide probes that are capable of binding specifically to genes encoding the heavy and light chains of the antibody). Expression is performed in appropriate prokaryotic or eukaryotic host cells like CHO cells, NS0 cells, SP2/0 cells, HEK293 cells, or COS cells, the antibody is recovered from the cells (supernatant or cells after lysis) by standard techniques. The antibodies are suitably separated from the culture medium by conventional immunoglobulin purification procedures such as, for example, column chromatography with protein ASepharose, hydroxylapatite chromatography, gel electrophoresis, dialysis, or affinity chromatography.

Amino Acid Sequences Disclosed in the Application:

SEQ ID NO:1 light chain, variable domain of HuMab 001

SEQ ID NO:2 heavy chain, variable domain of HuMab 001

SEQ ID NO:3 light chain, variable domain of HuMab 002

SEQ ID NO:4 heavy chain, variable domain of HuMab 002

SEQ ID NO:5 light chain, variable domain of HuMab 003

SEQ ID NO:6 heavy chain, variable domain of HuMab 003

SEQ ID NO:7 light chain (I), variable domain of HuMab 004 (I)

SEQ ID NO:8 heavy chain (I), variable domain of HuMab 004 (I)

SEQ ID NO:9 light chain (II), variable domain of HuMab 004 (II)

SEQ ID NO:10 heavy chain (II), variable domain of HuMab 004 (II)

SEQ ID NO:11 light chain, variable domain of HuMab 005

SEQ ID NO:12 heavy chain, variable domain of HuMab 005

SEQ ID NO:13 light chain, variable domain of HuMab 010 (I)

SEQ ID NO:14 heavy chain, variable domain of HuMab 010 (I)

SEQ ID NO:15 light chain, variable domain of HuMab 010 (II)

SEQ ID NO:16 heavy chain, variable domain of HuMab 010 (II)

SEQ ID NO:17 light chain, variable domain of HuMab 010 (III)

SEQ ID NO:18 heavy chain, variable domain of HuMab 010 (III)

SEQ ID NO:19 light chain, variable domain of HuMab 011

SEQ ID NO:20 heavy chain, variable domain of HuMab 011

SEQ ID NO:21 light chain, variable domain of HuMab 017

SEQ ID NO:22 heavy chain, variable domain of HuMab 017

SEQ ID NO 23 light chain of HuMab 002 IgG4v1

SEQ ID NO 24 heavy chain of HuMab 002 IgG4v1

### EXAMPLES

Liquid drug product formulations for intravenous (i.v.) administration according to the invention were developed as follows.

### Example 1

### Preparation of Liquid Formulations for Initial Formulation Screening

The huMAb P-selectin liquid formulations F1 to F24 as listed in Table 1 were prepared at a protein concentration of 50 mg/ml.

huMAb P-Selectin antibody prepared and obtained as described in WO2005/100402 was provided at a concentration of approximately 20 mg/mL in a 20 mM histidine buffer at a pH of approximately 5.5. The huMAb P-selectin antibody used in the examples is a human antibody comprising the heavy chain of SEQ ID NO:24 and the light chain of SEQ ID NO:23.

For the preparation of the liquid formulations huMAb P-Selectin was buffer-exchanged against a diafiltration buffer containing the anticipated buffer composition and concentrated by ultrafiltration to an antibody concentration of approximately 80 mg/mL. After completion of the ultrafiltration operation, the excipients (e.g. trehalose, methionine) were added as stock solutions to the antibody solution. The surfactant was then added as a 125-fold stock solution. Finally the protein concentration was adjusted with a buffer to the final huMAb P-Selectin concentration of approximately 50 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approx. 2.4 mL. These formulations were stored at different climate conditions (5° C., 25° C. and 40° C.) for different intervals of time and stressed by shaking (1 week at a shaking frequency of 200 min-1 at 5° C. and 25° C.) and freeze-thaw stress methods (five cycles at −80° C./+5° C.).

TABLE 1

| Code | Buffer | Surfactant | Excipient 1 | Excipient 2 |
|---|---|---|---|---|
| F1 | 20 mM | 0.04% Polysorbate 20 | 230 mM Trehalose | — |
| F2 | Histidine | 0.04% Polysorbate 20 | 230 mM Trehalose | 10 mM Methionine |
| F3 | Acetate pH 5.5 | 0.04% Polysorbate 20 | 210 mM Sucrose | — |
| F4 | | 0.04% Polysorbate 20 | 210 mM Sucrose | 10 mM Methionine |
| F5 | | 0.04% Polysorbate 20 | 145 mM Arginine | — |
| | | | Hydrochloride | |
| F6 | | 0.04% Polysorbate 20 | 145 mM Arginine | 10 mM Methionine |
| | | | Hydrochloride | |
| F7 | | 0.04% Polysorbate 20 | 230 mM Sorbitol | — |
| F8 | | 0.04% Polysorbate 20 | 230 mM Sorbitol | 10 mM Methionine |
| F9 | | 0.04% Polysorbate 80 | 230 mM Trehalose | — |
| F10 | | 0.04% Polysorbate 80 | 230 mM Trehalose | 10 mM Methionine |
| F11 | | 0.04% Polysorbate 80 | 210 mM Sucrose | — |
| F12 | | 0.04% Polysorbate 80 | 210 mM Sucrose | 10 mM Methionine |
| F13 | | 0.04% Polysorbate 80 | 145 mM Arginine | — |
| | | | Hydrochloride | |
| F14 | | 0.04% Polysorbate 80 | 145 mM Arginine | 10 mM Methionine |
| | | | Hydrochloride | |
| F15 | | 0.04% Polysorbate 80 | 230 mM Sorbitol | — |
| F16 | | 0.04% Polysorbate 80 | 230 mM Sorbitol | 10 mM Methionine |
| F17 | | 0.02% Poloxamer 188 | 230 mM Trehalose | — |
| F18 | | 0.02% Poloxamer 188 | 230 mM Trehalose | 10 mM Methionine |
| F19 | | 0.02% Poloxamer 188 | 210 mM Sucrose | — |
| F20 | | 0.02% Poloxamer 188 | 210 mM Sucrose | 10 mM Methionine |
| F21 | | 0.02% Poloxamer 188 | 145 mM Arginine | — |
| | | | Hydrochloride | |
| F22 | | 0.02% Poloxamer 188 | 145 mM Arginine | 10 mM Methionine |
| | | | Hydrochloride | |
| F23 | | 0.02% Poloxamer 188 | 230 mM Sorbitol | — |
| F24 | | 0.02% Poloxamer 188 | 230 mM Sorbitol | 10 mM Methionine |

The samples were analyzed before and after applying the stress tests as well as after storage by the following analytical methods:
UV spectroscopy
Size Exclusion Chromatography (SEC)
Ion exchange chromatography (IEC)
Clarity and opalescence of the solution
Analytical Protein A Chromatography
Visual inspection

UV spectroscopy, used for determination of protein content, was performed on a Perkin Elmer λ35 UV spectrophotometer in a wavelength range from 240 nm to 400 nm. Neat protein samples were diluted to approx. 0.5 mg/mL with the corresponding formulation buffer. The protein concentration was calculated according to equation 1.

$$Protein content = \frac{A ( 280 ) - A ( 320 ) \times dil \cdot factor}{ɛ 〈 \frac{{cm}^{2}}{mg} 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient ε.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2M K₂HPO₄/0.25M KCL, pH 7.0 as mobile phase, and were detected at a wavelength of 280 nm.

Ion Exchange Chromatography (IEC) was performed to detect chemical degradation products altering the net charge of huMAb P-Selectin in the formulations. The method used a Waters Alliance 2695 HPLC instrument with a Waters W2487 Dual Absorbance Detector and equipped (detection wavelength 280 nm) and a Dionex ProPac™ WCX-10, 4 mm×250 mm column. 10 mM Sodium Acetate, pH 5.8-5.9 and 1M Sodium Acetate, pH 5.8-5.9 are used as mobile phases A and B, respectively, at a flow rate of 1.0 mL/min.

Gradient program:

| | | % Mobile | % Mobile |
|---|---|---|---|
| | Time (min) | Phase A | Phase B |
| 1 | 0.01 | 100.0 | 0.0 |
| 2 | 5.00 | 100.0 | 0.0 |
| 3 | 45.00 | 85.0 | 15.0 |
| 4 | 65.00 | 47.0 | 53.0 |
| 5 | 67.00 | 0.0 | 100.0 |
| 6 | 72.00 | 0.0 | 100.0 |
| 7 | 74.00 | 100.0 | 0.0 |
| 8 | 95.00 | 100.0 | 0.0 |

Clarity and the degree of opalescence were measured as Formazine Turbidity Units (FTU) by the method of nephelometry. The neat sample was transferred into a 11 mm diameter clearglass tube and placed into a HACH 2100AN turbidimeter.

Analytical Protein A chromatography was performed to monitor the oxidation status of the four conserved methionine side chains in the Fc part of huMab P-Selectin. The method was performed on a Waters Alliance 2695 HPLC instrument with a Waters W2487 Dual Absorbance Detector (detection wavelength 280 nm), equipped with a Poros A/20 4.6 mm×50 mm column from Applied Biosystems, USA. PBS from Gibco, Invitrogen and 0.1M acetic acid, 0.15M sodium chloride, pH 2.9 were used as mobile phases A and B, respectively, at a flow rate of 2.0 mL/min:

Gradient program:

| | | % Mobile | % Mobile |
|---|---|---|---|
| | Time (min) | Phase A | Phase B |
| 1 | 0.01 | 100 | 0 |
| 2 | 10 | 100 | 0 |
| 3 | 40 | 40 | 60 |
| 4 | 41 | 0 | 100 |
| 5 | 51 | 0 | 100 |
| 6 | 52 | 100 | 0 |
| 7 | 62 | 100 | 0 |

Samples were inspected for the presence of visible particles by using a Seidenader V90-T visual inspection instrument.

| Number of Particles | |
|---|---|
| per Vial | Description |
| 0 | Free from Particles |
| 1-5 | Essentially free from Particles |
| 6-10 | With a few Particles |
| >10 | With many Particles |

### Example 1

### Compositions and Stability Data of Liquid huMAb P-Selectin Drug Product Formulations According to this Invention

F1 is a liquid formulation with the composition 50 mg/mL huMAb P-Selectin, 20 mM histidine acetate, 230 mM trehalose, 0.04% polysorbate 20, at pH 5.5

| | | | | | | | Ion Exchange-HPLC | | | | |
|---|---|---|---|---|---|---|---|---|---|---|---|
| | | | Protein | Size Exclusion-HPLC | | | | Acidic | Basic | | |
| Storage | Storage | | conc. | HMW | Monomer | LMW | Main Peak | Species | species | Turbidity | |
| condition | Time | | (mg/mL) | (%) | (%) | (%) | (%) | (%) | (%) | (FTU) | Visible particles |
| — | Initial | | 47.7 | 0.9 | 98.6 | 0.4 | 71.5 | 11.9 | 16.7 | 9.0 | Free from Particles |
| Shaking 5° C. | 1 | week | 48.1 | 0.9 | 98.7 | 0.4 | 70.3 | 11.8 | 17.9 | 9.1 | Free from Particles |
| Shaking 25° C. | 1 | week | 48.0 | 0.9 | 98.7 | 0.4 | 7202 | 11.5 | 16.2 | 8.7 | Free from Particles |
| Freeze/thaw | 5 | cycles | 47.8 | 0.9 | 98.7 | 0.4 | 71.9 | 12.0 | 16.1 | 9.5 | Free from Particles |
| 5° C. | 9 | months | 47.8 | 1.1 | 98.3 | 0.6 | 71.0 | 11.7 | 17.4 | 9.6 | Free from Particles |
| 25° C. | 9 | months | — | 2.3 | 96.7 | 1.0 | 59.8 | 18.0 | 22.3 | 9.9 | Free from Particles |
| 40° C. | 9 | months | — | 34.9 | 61.0 | 4.1 | — | — | — | 21.1 | Free from Particles |

| | Storage | Analytical Protein A Chromatography |
|---|---|---|
| Storage condition | Time | Fc Methionine Oxidation (%) |
| — | Initial | 1.9 |
| 5° C. | 9 months | 4.5 |
| 25° C. | 9 months | 40.6 |

F2 is a liquid formulation with the composition 50 mg/mL huMAb P-Selectin, 20 mM histidine acetate, 230 mM trehalose, 0.04% polysorbate 20, 10 mM Methionine at pH 5.5

| | | | | | | Ion Exchange-HPLC | | | | |
|---|---|---|---|---|---|---|---|---|---|---|
| | | Protein | Size Exclusion-HPLC | | | | Acidic | Basic | | |
| | Storage | conc. | HMW | Monomer | LMW | Main Peak | Species | species | Turbidity | |
| Storage condition | Time | (mg/mL) | (%) | (%) | (%) | (%) | (%) | (%) | (FTU) | Visible particles |
| — | Initial | 47.8 | 0.9 | 98.7 | 0.4 | 72.5 | 11.6 | 15.9 | 8.4 | Free from Particles |
| Shaking 5° C. | 1 week | 48.0 | 0.9 | 98.8 | 0.4 | 70.4 | 11.8 | 17.8 | 8.4 | Free from Particles |
| Shaking 25° C. | 1 week | 48.5 | 0.9 | 98.8 | 0.3 | 71.7 | 11.7 | 16.6 | 8.9 | Free from Particles |
| Freeze/thaw | 5 cycles | 48.7 | 0.9 | 98.8 | 0.4 | 72.1 | 12.0 | 15.9 | 8.7 | Free from Particles |
| 5° C. | 9 months | 47.2 | 1.0 | 98.3 | 0.6 | 71.1 | 11.7 | 17.2 | 9.7 | Free from Particles |
| 25° C. | 9 months | — | 1.4 | 97.7 | 0.8 | 65.6 | 15.7 | 18.8 | 9.1 | Free from Particles |
| 40° C. | 9 months | — | 21.0 | 75.5 | 3.5 | — | — | — | 16 | Free from Particles |

| | Storage | Analytical Protein A Chromatography |
|---|---|---|
| Storage condition | Time | Fc Methionine Oxidation (%) |
| — | Initial | 1.9 |
| 5° C. | 9 months | 3.6 |
| 25° C. | 9 months | 4.4 |

F3 is a liquid formulation with the composition 50 mg/mL huMAb P-Selectin, 20 mM histidine acetate, 210 mM Sucrose, 0.04% polysorbate 20, at pH 5.5

| | | | | | | Ion Exchange-HPLC | | | | |
|---|---|---|---|---|---|---|---|---|---|---|
| | | Protein | Size Exclusion-HPLC | | | | Acidic | Basic | | |
| | Storage | conc. | HMW | Monomer | LMW | Main Peak | Species | species | Turbidity | |
| Storage condition | Time | (mg/mL) | (%) | (%) | (%) | (%) | (%) | (%) | (FTU) | Visible particles |
| — | Initial | 47.7 | 0.9 | 98.7 | 0.4 | 72.5 | 11.6 | 16.0 | 9.0 | Free from Particles |
| Shaking 5° C. | 1 week | 48.2 | 0.9 | 98.8 | 0.3 | 70.0 | 11.8 | 18.1 | 9.0 | Essentially Free |
| | | | | | | | | | | from Particles |
| Shaking 25° C. | 1 week | 48.2 | 0.9 | 98.7 | 0.3 | 72.0 | 11.7 | 16.4 | 9.7 | Free from Particles |
| Freeze/thaw | 5 cycles | 48.4 | 0.9 | 98.7 | 0.3 | 70.5 | 11.8 | 17.7 | 9.3 | Free from Particles |
| 5° C. | 9 months | 47.7 | 1.1 | 98.2 | 0.7 | 70.5 | 11.6 | 17.9 | 9.4 | Free from Particles |
| 25° C. | 9 months | — | 2.0 | 97.0 | 1.0 | 61.9 | 17.2 | 20.9 | 10.2 | Free from Particles |
| 40° C. | 9 months | — | 40.4 | 56.0 | 3.6 | — | — | — | 23.9 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.0 |
| 25° C. | 9 months | 33.9 |

F4 is a liquid formulation with the composition 50 mg/mL huMAb P-Selectin, 20 mM histidine acetate, 210 mM Sucrose, 0.04% polysorbate 20, 10 mM Methionine at pH 5.5

| | | | | | | Ion Exchange-HPLC | | | | |
|---|---|---|---|---|---|---|---|---|---|---|
| | | Protein | Size Exclusion-HPLC | | | | Acidic | Basic | | |
| | Storage | conc. | HMW | Monomer | LMW | Main Peak | Species | species | Turbidity | |
| Storage condition | Time | (mg/mL) | (%) | (%) | (%) | (%) | (%) | (%) | (FTU) | Visible particles |
| — | Initial | 48.4 | 0.9 | 98.7 | 0.4 | 72.2 | 11.5 | 16.2 | 8.5 | Free from Particles |
| Shaking 5° C. | 1 week | 48.2 | 0.9 | 98.8 | 0.3 | 70.5 | 11.8 | 17.6 | 9.1 | Free from Particles |
| Shaking 25° C. | 1 week | 48.1 | 0.9 | 98.7 | 0.4 | 71.8 | 11.6 | 16.6 | 9.4 | Free from Particles |
| Freeze/thaw | 5 cycles | 48.1 | 0.9 | 98.8 | 0.4 | 71.2 | 11.8 | 17.0 | 9.5 | Essentially Free |
| | | | | | | | | | | from Particles |
| 5° C. | 9 months | 47.8 | 1.0 | 98.3 | 0.6 | 71.3 | 11.5 | 17.2 | 10.0 | Free from Particles |
| 25° C. | 9 months | — | 1.4 | 97.7 | 0.9 | 66.6 | 15.5 | 17.9 | 9.4 | Free from Particles |
| 40° C. | 9 months | — | 24.4 | 72.0 | 3.6 | — | — | — | 17.5 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.0 |
| 25° C. | 9 months | 4.3 |

F5 is a liquid formulation with the composition 50 mg/mL huMAb P-Selectin, 20 mM histidine acetate, 145 mM Arginine Hydrochloride, 0.04% polysorbate 20, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.6 | 0.9 | 98.7 | 0.4 | 72.1 | 11.5 | 16.4 | 16.3 | Essentially Free |
| | | | | | | | | | | from Particles |
| Shaking 5° C. | 1 week | 49.3 | 0.9 | 98.8 | 0.3 | 70.6 | 11.8 | 17.7 | 17.0 | Free from Particles |
| Shaking 25° C. | 1 week | 49.8 | 0.9 | 98.8 | 0.3 | 72.3 | 11.5 | 16.2 | 17.8 | Free from Particles |
| Freeze/thaw | 5 cycles | 49.0 | 0.9 | 98.8 | 0.3 | 71.7 | 11.6 | 16.7 | 17.1 | Free from Particles |
| 5° C. | 9 months | 48.9 | 1.0 | 98.3 | 0.6 | 70.8 | 11.5 | 17.7 | 17.2 | Free from Particles |
| 25° C. | 9 months | — | 2.1 | 96.9 | 1.1 | 60.7 | 15.7 | 23.6 | 17.4 | Essentially Free |
| | | | | | | | | | | from Particles |
| 40° C. | 9 months | — | 37.1 | 58.9 | 4.0 | — | — | — | 78.6 | Free from Particles |

| | Storage | Analytical Protein A Chromatography |
|---|---|---|
| Storage condition | Time | Fc Methionine Oxidation (%) |
| — | Initial | 2.2 |
| 5° C. | 9 months | 4.0 |
| 25° C. | 9 months | 39.3 |

F6 is a liquid formulation with the composition 50 mg/mL huMAb P-Selectin, 20 mM histidine acetate, 145 mM Arginine Hydrochloride, 0.04% polysorbate 20, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.4 | 0.9 | 98.7 | 0.4 | 72.6 | 11.5 | 15.9 | 16.7 | Essentially Free |
| | | | | | | | | | | from Particles |
| Shaking 5° C. | 1 week | 48.9 | 0.8 | 98.9 | 0.3 | 70.5 | 11.7 | 17.7 | 17.5 | Free from Particles |
| Shaking 25° C. | 1 week | 48.0 | 0.8 | 98.8 | 0.3 | 71.7 | 11.5 | 16.8 | 17.2 | Free from Particles |
| Freeze/thaw | 5 cycles | 49.6 | 0.8 | 98.8 | 0.4 | 71.2 | 11.8 | 16.9 | 17.4 | Free from Particles |
| 5° C. | 9 months | 49.1 | 1.0 | 98.5 | 0.6 | 70.8 | 11.5 | 17.7 | 16.8 | Free from Particles |
| 25° C. | 9 months | — | 1.3 | 97.9 | 0.9 | 66.3 | 14.8 | 18.9 | 17.4 | Free from Particles |
| 40° C. | 9 months | — | 24.7 | 71.6 | 3.6 | — | — | — | 43.2 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 3.9 |
| 25° C. | 9 months | 4.3 |

F7 is a liquid formulation with the composition 50 mg/mL huMAb P-Selectin, 20 mM histidine acetate, 230 mM sorbitol, 0.04% polysorbate 20, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.3 | 0.9 | 98.7 | 0.4 | 72.5 | 11.6 | 15.9 | 10.0 | Free from Particles |
| Shaking 5° C. | 1 week | 48.7 | 0.9 | 98.8 | 0.3 | 70.5 | 11.9 | 17.6 | 10.2 | Free from Particles |
| Shaking 25° C. | 1 week | 49.0 | 1.0 | 98.7 | 0.4 | 71.9 | 11.8 | 16.3 | 10.5 | Free from Particles |
| Freeze/thaw | 5 cycles | 49.2 | 0.9 | 98.8 | 0.3 | 71.6 | 11.9 | 16.5 | 10.3 | Essentially Free |
| | | | | | | | | | | from Particles |
| 5° C. | 9 months | 48.3 | 1.1 | 98.3 | 0.6 | 70.5 | 11.8 | 17.7 | 10.6 | Essentially Free |
| | | | | | | | | | | from Particles |
| 25° C. | 9 months | — | 2.1 | 97.0 | 1.0 | 61.2 | 17.3 | 21.5 | 11.2 | Free from Particles |
| 40° C. | 9 months | — | 31.5 | 64.5 | 4.0 | — | — | — | 23.4 | Free from Particles |

| | Storage | Analytical Protein A Chromatography |
|---|---|---|
| Storage condition | Time | Fc Methionine Oxidation (%) |
| — | Initial | 2.2 |
| 5° C. | 9 months | 3.9 |
| 25° C. | 9 months | 32.6 |

F8 is a liquid formulation with the composition 50 mg/mL huMAb P-Selectin, 20 mM histidine acetate, 230 mM sorbitol, 0.04% polysorbate 20, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.3 | 0.9 | 98.7 | 0.4 | 72.5 | 11.7 | 15.9 | 9.6 | Free from Particles |
| Shaking 5° C. | 1 week | 48.9 | 0.9 | 98.8 | 0.4 | 70.4 | 11.9 | 17.7 | 9.7 | Free from Particles |
| Shaking 25° C. | 1 week | 49.4 | 0.9 | 98.7 | 0.3 | 72.5 | 11.6 | 15.9 | 9.5 | Free from Particles |
| Freeze/thaw | 5 cycles | 49.1 | 0.9 | 98.8 | 0.3 | 71.5 | 11.9 | 16.6 | 10.1 | Essentially Free |
| | | | | | | | | | | from Particles |
| 5° C. | 9 months | 48.6 | 1.0 | 98.4 | 0.6 | 70.3 | 11.7 | 18.1 | 10.9 | Free from Particles |
| 25° C. | 9 months | — | 1.5 | 97.7 | 0.8 | 65.1 | 16.0 | 18.9 | 11.3 | Free from Particles |
| 40° C. | 9 months | — | 23.2 | 73.4 | 3.4 | — | — | — | 19 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 3.7 |
| 25° C. | 9 months | 4.4 |

F9 is a liquid formulation with the composition 50 mg/mL huMAb P-Selectin, 20 mM histidine acetate, 230 mM trehalose, 0.04% polysorbate 80, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.5 | 1.0 | 98.6 | 0.4 | 71.5 | 11.6 | 17.0 | 9.0 | Free from Particles |
| Shaking 5° C. | 1 week | 48.4 | 0.9 | 98.8 | 0.3 | 70.6 | 11.9 | 17.5 | 9.0 | Free from Particles |
| Shaking 25° C. | 1 week | 48.5 | 1.0 | 98.7 | 0.4 | 72.5 | 11.6 | 15.9 | 9.3 | Free from Particles |
| Freeze/thaw | 5 cycles | 48.3 | 0.9 | 98.7 | 0.4 | 71.7 | 11.7 | 16.6 | 9.2 | Essentially Free |
| | | | | | | | | | | from Particles |
| 5° C. | 9 months | 48.0 | 1.1 | 98.3 | 0.6 | 69.8 | 11.9 | 18.4 | 10.0 | Free from Particles |
| 25° C. | 9 months | — | 2.3 | 96.5 | 1.3 | 58.1 | 19.8 | 22.1 | 9.8 | Free from Particles |
| 40° C. | 9 months | — | 36.7 | 59.8 | 3.8 | — | — | — | 21.1 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 5.0 |
| 25° C. | 9 months | 40.2 |

F10 is a liquid formulation with the composition 50 mg/mL huMAb P-Selectin, 20 mM histidine acetate, 230 mM trehalose, 0.04% polysorbate 80, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.1 | 0.9 | 98.7 | 0.4 | 71.6 | 11.5 | 16.9 | 8.5 | Free from Particles |
| Shaking 5° C. | 1 week | 47.8 | 0.9 | 98.8 | 0.4 | 70.7 | 11.8 | 17.5 | 9.5 | Free from Particles |
| Shaking 25° C. | 1 week | 48.1 | 0.9 | 98.7 | 0.4 | 72.1 | 11.5 | 16.4 | 9.0 | Free from Particles |
| Freeze/thaw | 5 cycles | 48.1 | 0.9 | 98.8 | 0.4 | 71.6 | 11.8 | 16.6 | 8.7 | Free from Particles |
| 5° C. | 9 months | 47.4 | 1.1 | 98.4 | 0.6 | 70.7 | 11.6 | 17.7 | 9.7 | Free from Particles |
| 25° C. | 9 months | — | 1.5 | 97.6 | 0.9 | 64.3 | 16.9 | 18.8 | 9.8 | Free from Particles |
| 40° C. | 9 months | — | 22.2 | 74.2 | 3.7 | — | — | — | 16.9 | Essentially Free |
| | | | | | | | | | | from Particles |

| | Storage | Analytical Protein A Chromatography |
|---|---|---|
| Storage condition | Time | Fc Methionine Oxidation (%) |
| — | Initial | 2.0 |
| 5° C. | 9 months | 4.1 |
| 25° C. | 9 months | 5.4 |

F11 is a liquid formulation with the composition 50 mg/mL huMAb P-Selectin, 20 mM histidine acetate, 210 mM Sucrose, 0.04% polysorbate 80, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.2 | 1.0 | 98.6 | 0.4 | 72.0 | 11.6 | 16.4 | 8.5 | Free from Particles |
| Shaking 5° C. | 1 week | 48.1 | 0.9 | 98.8 | 0.3 | 70.2 | 11.8 | 18.0 | 8.7 | Free from Particles |
| Shaking 25° C. | 1 week | 48.7 | 1.0 | 98.7 | 0.4 | 71.3 | 11.7 | 17.0 | 9.5 | Essentially Free |
| | | | | | | | | | | from Particles |
| Freeze/thaw | 5 cycles | 48.5 | 0.9 | 98.7 | 0.3 | 71.3 | 12.0 | 16.8 | 9.5 | Free from Particles |
| 5° C. | 9 months | 48.2 | 1.1 | 98.3 | 0.6 | 70.5 | 11.8 | 17.7 | 9.5 | Free from Particles |
| 25° C. | 9 months | — | 2.4 | 96.5 | 1.2 | 58.0 | 19.9 | 22.1 | 9.4 | Free from Particles |
| 40° C. | 9 months | — | 43.1 | 53.6 | 3.3 | — | — | — | 24.1 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.7 |
| 25° C. | 9 months | 39.1 |

F12 is a liquid formulation with the composition 50 mg/mL huMAb P-Selectin, 20 mM histidine acetate, 210 mM Sucrose, 0.04% polysorbate 80, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.4 | 0.9 | 98.7 | 0.4 | 72.0 | 11.6 | 16.4 | 8.6 | Free from Particles |
| Shaking 5° C. | 1 week | 48.3 | 0.9 | 98.7 | 0.4 | 70.3 | 11.8 | 17.9 | 9.3 | Essentially Free |
| | | | | | | | | | | from Particles |
| Shaking 25° C. | 1 week | 48.7 | 0.9 | 98.7 | 0.4 | 71.4 | 11.6 | 17.0 | 9.2 | Free from Particles |
| Freeze/thaw | 5 cycles | 48.5 | 0.9 | 98.8 | 0.4 | 71.4 | 11.8 | 16.8 | 9.3 | Free from Particles |
| 5° C. | 9 months | 48.2 | 1.1 | 98.3 | 0.6 | 70.4 | 11.7 | 17.9 | 10.5 | Free from Particles |
| 25° C. | 9 months | — | 1.5 | 97.6 | 0.9 | 64.0 | 17.0 | 19.0 | 9.6 | Free from Particles |
| 40° C. | 9 months | — | 25.6 | 70.8 | 3.7 | — | — | — | 17.7 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.0 |
| 25° C. | 9 months | 5.2 |

F13 is a liquid formulation with the composition 50 mg/mL huMab P-Selectin, 20 mM histidine acetate, 145 mM Arginine Hydrochloride, 0.04% polysorbate 80, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.3 | 1.0 | 98.6 | 0.4 | 72.0 | 11.5 | 16.5 | 16.3 | Free from Particles |
| Shaking 5° C. | 1 week | 48.9 | 0.9 | 98.8 | 0.3 | 70.1 | 11.8 | 18.1 | 17.1 | Free from Particles |
| Shaking 25° C. | 1 week | 49.2 | 0.9 | 98.8 | 0.3 | 71.9 | 11.5 | 17.0 | 16.7 | Free from Particles |
| Freeze/thaw | 5 cycles | 49.1 | 0.9 | 98.8 | 0.4 | 71.1 | 11.9 | 17.0 | 16.9 | Free from Particles |
| 5° C. | 9 months | 48.3 | 1.1 | 98.3 | 0.7 | 70.2 | 11.5 | 18.4 | 17.4 | Free from Particles |
| 25° C. | 9 months | — | 2.2 | 96.6 | 1.2 | 60.0 | 16.3 | 23.7 | 17.6 | Free from Particles |
| 40° C. | 9 months | — | 38.7 | 57.4 | 4.0 | — | — | — | 78.3 | Essentially Free |
| | | | | | | | | | | from Particles |

| | Storage | Analytical Protein A Chromatography |
|---|---|---|
| Storage condition | Time | Fc Methionine Oxidation (%) |
| — | Initial | 2.2 |
| 5° C. | 9 months | 5.7 |
| 25° C. | 9 months | 38.0 |

F14 is a liquid formulation with the composition 50 mg/mL huMAb P-Selectin, 20 mM histidine acetate, 145 mM Arginine Hydrochloride, 0.04% polysorbate 80, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.7 | 0.9 | 98.7 | 0.4 | 72.1 | 11.5 | 16.4 | 15.8 | Free from Particles |
| Shaking 5° C. | 1 week | 49.0 | 0.8 | 98.8 | 0.3 | 70.5 | 11.8 | 17.7 | 15.5 | Essentially Free |
| | | | | | | | | | | from Particles |
| Shaking 25° C. | 1 week | 49.4 | 0.9 | 98.8 | 0.3 | 71.8 | 11.5 | 16.7 | 16.0 | Essentially Free |
| | | | | | | | | | | from Particles |
| Freeze/thaw | 5 cycles | 49.1 | 0.8 | 98.8 | 0.3 | 70.9 | 11.6 | 17.5 | 16.4 | Free from Particles |
| 5° C. | 9 months | 48.4 | 1.0 | 98.4 | 0.6 | 70.5 | 11.5 | 18.0 | 17.2 | Free from Particles |
| 25° C. | 9 months | — | 1.3 | 97.7 | 0.9 | 65.2 | 15.2 | 19.7 | 16.4 | Free from Particles |
| 40° C. | 9 months | — | 25.9 | 70.3 | 3.8 | — | — | — | 46.3 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.2 |
| 25° C. | 9 months | 5.1 |

F15 is a liquid formulation with the composition 50 mg/mL huMab P-Selectin, 20 mM histidine acetate, 230 mM sorbitol, 0.04% polysorbate 80, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.6 | 1.0 | 98.6 | 0.4 | 72.1 | 11.6 | 16.4 | 9.8 | Free from Particles |
| Shaking 5° C. | 1 week | 48.9 | 0.9 | 98.7 | 0.3 | 70.1 | 11.9 | 18.0 | 10.5 | Essentially Free |
| | | | | | | | | | | from Particles |
| Shaking 25° C. | 1 week | 48.9 | 1.0 | 98.7 | 0.3 | 71.6 | 11.7 | 16.7 | 1.1 | Free from Particles |
| Freeze/thaw | 5 cycles | 49.8 | 1.0 | 98.7 | 0.3 | 71.8 | 11.9 | 16.3 | 10.2 | Free from Particles |
| 5° C. | 9 months | 47.6 | 1.1 | 98.3 | 0.6 | 69.9 | 12.0 | 18.2 | 10.7 | Free from Particles |
| 25° C. | 9 months | — | 2.4 | 96.4 | 1.2 | 59.3 | 19.5 | 21.2 | 10.2 | Free from Particles |
| 40° C. | 9 months | — | 34.9 | 61.3 | 3.8 | — | — | — | 23.4 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.7 |
| 25° C. | 9 months | 37.8 |

F16 is a liquid formulation with the composition 50 mg/mL huMAb P-Selectin, 20 mM histidine acetate, 230 mM sorbitol, 0.04% polysorbate 80, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 49.9 | 1.0 | 98.7 | 0.4 | 72.1 | 11.7 | 16.3 | 9.3 | Free from Particles |
| Shaking 5° C. | 1 week | 49.7 | 0.9 | 98.8 | 0.3 | 70.5 | 11.9 | 17.6 | 10.3 | Free from Particles |
| Shaking 25° C. | 1 week | 49.5 | 1.0 | 98.7 | 0.4 | 71.7 | 11.8 | 16.6 | 10.4 | Free from Particles |
| Freeze/thaw | 5 cycles | 49.8 | 0.9 | 98.7 | 0.3 | 71.4 | 11.9 | 16.8 | 9.8 | Free from Particles |
| 5° C. | 9 months | 48.5 | 1.1 | 98.3 | 0.7 | 69.9 | 11.9 | 18.2 | 10.7 | Free from Particles |
| 25° C. | 9 months | — | 1.6 | 97.6 | 0.9 | 64.1 | 17.3 | 18.6 | 11.7 | Free from Particles |
| 40° C. | 9 months | — | 24.5 | 72.1 | 3.5 | — | — | — | 19.3 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.0 |
| 25° C. | 9 months | 5.3 |

F17 is a liquid formulation with the composition 50 mg/mL huMAb P-Selectin, 20 mM histidine acetate, 230 mM trehalose, 0.02% Poloxamer 188, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.6 | 0.9 | 98.7 | 0.4 | 72.2 | 11.6 | 16.3 | 8.6 | Free from Particles |
| Shaking 5° C. | 1 week | 48.3 | 0.9 | 98.7 | 0.4 | 70.3 | 11.8 | 17.9 | 9.7 | Free from Particles |
| Shaking 25° C. | 1 week | 48.2 | 1.0 | 98.7 | 0.3 | 71.8 | 11.6 | 16.5 | 9.3 | Free from Particles |
| Freeze/thaw | 5 cycles | 48.0 | 0.9 | 98.7 | 0.4 | 71.4 | 11.8 | 16.9 | 9.8 | Free from Particles |
| 5° C. | 9 months | 47.8 | 1.1 | 98.3 | 0.6 | 70.0 | 11.8 | 18.2 | 9.5 | Free from Particles |
| 25° C. | 9 months | — | 1.6 | 97.6 | 0.8 | 64.5 | 16.0 | 19.4 | 9.8 | Free from Particles |
| 40° C. | 9 months | — | 24.6 | 71.1 | 4.3 | — | — | — | 17.5 | Free from Particles |

| | Storage | Analytical Protein A Chromatography |
|---|---|---|
| Storage condition | Time | Fc Methionine Oxidation (%) |
| — | Initial | 2.2 |
| 5° C. | 9 months | 4.1 |
| 25° C. | 9 months | 5.1 |

F18 is a liquid formulation with the composition 50 mg/mL huMAb P-Selectin, 20 mM histidine acetate, 230 mM trehalose, 0.02% Poloxamer 188, 10 mM Methionine at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.6 | 0.9 | 98.6 | 0.4 | 72.1 | 11.6 | 16.3 | 8.6 | Essentially Free |
| | | | | | | | | | | from Particles |
| Shaking 5° C. | 1 week | 47.9 | 0.9 | 98.8 | 0.3 | 70.4 | 11.8 | 17.7 | 9.2 | Free from Particles |
| Shaking 25° C. | 1 week | 48.0 | 0.9 | 98.7 | 0.3 | 71.7 | 11.7 | 16.6 | 8.7 | Free from Particles |
| Freeze/thaw | 5 cycles | 48.2 | 0.9 | 98.7 | 0.4 | 71.3 | 12.0 | 16.8 | 9.1 | Essentially Free |
| | | | | | | | | | | from Particles |
| 5° C. | 9 months | 47.6 | 1.0 | 98.4 | 0.6 | 70.7 | 11.7 | 17.6 | 9.7 | Free from Particles |
| 25° C. | 9 months | — | 1.4 | 97.8 | 0.8 | 65.8 | 15.7 | 18.4 | 9.3 | Essentially Free |
| | | | | | | | | | | from Particles |
| 40° C. | 9 months | — | 20.0 | 76.5 | 3.5 | — | — | — | 15.5 | Free from Particles |

| | Storage | Analytical Protein A Chromatography |
|---|---|---|
| Storage condition | Time | Fc Methionine Oxidation (%) |
| — | Initial | 2.1 |
| 5° C. | 9 months | 3.7 |
| 25° C. | 9 months | 4.0 |

F19 is a liquid formulation with the composition 50 mg/mL huMab P-Selectin, 20 mM histidine acetate, 210 mM Sucrose, 0.02% Poloxamer 188, at pH 5.5

| | | Protein | Size Exclusion-HPLC | | | Ion Exchange-HPLC | | | | |
|---|---|---|---|---|---|---|---|---|---|---|
| | Storage | conc. | HMW | Monomer | LMW | Main Peak | Acidic Species | Basic species | Turbidity | |
| Storage condition | Time | (mg/mL) | (%) | (%) | (%) | (%) | (%) | (%) | (FTU) | Visible particles |
| — | Initial | 48.1 | 0.9 | 98.6 | 0.4 | 71.7 | 11.6 | 16.8 | 8.7 | Essentially Free |
| | | | | | | | | | | from Particles |
| Shaking 5° C. | 1 week | 48.5 | 0.9 | 98.8 | 0.3 | 70.7 | 11.9 | 17.4 | 9.8 | Essentially Free |
| | | | | | | | | | | from Particles |
| Shaking 25° C. | 1 week | 48.6 | 0.9 | 98.7 | 0.3 | 72.6 | 11.5 | 16.0 | 9.6 | Free from Particles |
| Freeze/thaw | 5 cycles | 48.6 | 0.9 | 98.7 | 0.4 | 71.2 | 11.8 | 17.0 | 9.6 | Free from Particles |
| 5° C. | 9 months | 48.7 | 1.1 | 98.3 | 0.6 | 69.8 | 11.9 | 18.3 | 9.8 | Free from Particles |
| 25° C. | 9 months | — | 1.6 | 97.5 | 0.8 | 64.8 | 16.2 | 19.1 | 10.2 | Free from Particles |
| 40° C. | 9 months | — | 32.7 | 63.5 | 3.8 | — | — | — | 18.8 | Free from Particles |

| | Storage | Analytical Protein A Chromatography |
|---|---|---|
| Storage condition | Time | Fc Methionine Oxidation (%) |
| — | Initial | 2.3 |
| 5° C. | 9 months | 4.0 |
| 25° C. | 9 months | 4.9 |

F20 is a liquid formulation with the composition 50 mg/mL huMAb P-Selectin, 20 mM histidine acetate, 210 mM Sucrose, 0.02% Poloxamer 188, 10 mM Methionine at pH 5.5

| | | | | | | Ion Exchange-HPLC | | | | |
|---|---|---|---|---|---|---|---|---|---|---|
| | | Protein | Size Exclusion-HPLC | | | Main | Acidic | | | |
| | Storage | conc. | HMW | Monomer | LMW | Peak | Species | Basic species | Turbidity | |
| Storage condition | Time | (mg/mL) | (%) | (%) | (%) | (%) | (%) | (%) | (FTU) | Visible particles |
| — | Initial | 48.5 | 0.9 | 98.7 | 0.4 | 72.1 | 11.6 | 16.3 | 8.7 | Free from Particles |
| Shaking 5° C. | 1 week | 48.3 | 0.9 | 98.8 | 0.3 | 70.7 | 11.9 | 17.4 | 9.5 | Free from Particles |
| Shaking 25° C. | 1 week | 48.8 | 0.9 | 98.8 | 0.3 | 71.5 | 11.8 | 16.7 | 9.3 | Free from Particles |
| Freeze/thaw | 5 cycles | 48.4 | 0.9 | 98.8 | 0.4 | 71.3 | 11.8 | 16.9 | 9.3 | Free from Particles |
| 5° C. | 9 months | 48.0 | 1.0 | 98.3 | 0.6 | 70.4 | 11.6 | 18.1 | 9.5 | Free from Particles |
| 25° C. | 9 months | — | 1.4 | 97.8 | 0.8 | 66.2 | 15.5 | 18.3 | 9.7 | Free from Particles |
| 40° C. | 9 months | — | 23.3 | 73.1 | 3.7 | — | — | — | 16.0 | Free from Particles |

| | Storage | Analytical Protein A Chromatography |
|---|---|---|
| Storage condition | Time | Fc Methionine Oxidation (%) |
| — | Initial | 2.2 |
| 5° C. | 9 months | 3.8 |
| 25° C. | 9 months | 4.3 |

F21 is a liquid formulation with the composition 50 mg/mL huMAb P-Selectin, 20 mM histidine acetate, 145 mM Arginine Hydrochloride, 0.02% Poloxamer 188, at pH 5.5

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 50.3 | 0.9 | 98.7 | 0.4 | 72.1 | 11.5 | 16.3 | 19.6 | Free from Particles |
| Shaking 5° C. | 1 week | 50.3 | 0.9 | 98.8 | 0.3 | 70.8 | 11.8 | 17.5 | 17.6 | Free from Particles |
| Shaking 25° C. | 1 week | 50.1 | 0.9 | 98.8 | 0.3 | 72.0 | 11.6 | 16.5 | 17.6 | Free from Particles |
| Freeze/thaw | 5 cycles | 50.0 | 0.9 | 98.8 | 0.3 | 72.0 | 11.5 | 16.5 | 18.6 | Free from Particles |
| 5° C. | 9 months | 49.9 | 1.1 | 98.3 | 0.6 | 70.3 | 11.6 | 18.1 | 18.5 | Free from Particles |
| 25° C. | 9 months | — | 1.4 | 97.8 | 0.8 | 65.5 | 14.9 | 19.6 | 18.8 | Free from Particles |
| 40° C. | 9 months | — | 26.3 | 69.7 | 4.1 | — | — | — | 47.3 | Essentially Free from |
| | | | | | | | | | | Particles |

| | Storage | Analytical Protein A Chromatography |
|---|---|---|
| Storage condition | Time | Fc Methionine Oxidation (%) |
| — | Initial | 2.4 |
| 5° C. | 9 months | 4.2 |
| 25° C. | 9 months | 5.0 |

F22 is a liquid formulation with the composition 50 mg/mL huMab P-Selectin, 20 mM histidine acetate, 145 mM Arginine Hydrochloride, 0.02% Poloxamer 188, 10 mM Methionine at pH 5.5

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 50.2 | 0.9 | 98.7 | 0.4 | 72.0 | 11.5 | 16.5 | 16.4 | Essentially Free from |
| | | | | | | | | | | Particles |
| Shaking 5° C. | 1 week | 50.4 | 0.8 | 98.9 | 0.3 | 70.4 | 11.8 | 17.8 | 16.4 | Free from Particles |
| Shaking 25° C. | 1 week | 50.1 | 0.9 | 98.8 | 0.3 | 72.2 | 11.6 | 16.2 | 17.0 | Free from Particles |
| Freeze/thaw | 5 cycles | 50.1 | 0.8 | 98.8 | 0.3 | 71.2 | 11.9 | 16.9 | 17.9 | Free from Particles |
| 5° C. | 9 months | 49.6 | 1.0 | 98.4 | 0.6 | 70.2 | 11.6 | 18.3 | 17.8 | Free from Particles |
| 25° C. | 9 months | — | 1.3 | 98.0 | 0.8 | 66.5 | 14.5 | 19.0 | 16.7 | Free from Particles |
| 40° C. | 9 months | — | 23.6 | 72.7 | 3.7 | — | — | — | 42.5 | Essentially Free from |
| | | | | | | | | | | Particles |

| | Storage | Analytical Protein A Chromatography |
|---|---|---|
| Storage condition | Time | Fc Methionine Oxidation (%) |
| — | Initial | 2.2 |
| 5° C. | 9 months | 3.8 |
| 25° C. | 9 months | 4.3 |

F23 is a liquid formulation with the composition 50 mg/mL huMab P-Selectin, 20 mM histidine acetate, 230 mM sorbitol, 0.02% Poloxamer 188, at pH 5.5

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 49.2 | 1.0 | 98.7 | 0.4 | 70.9 | 11.6 | 17.5 | 9.4 | Free from Particles |
| Shaking 5° C. | 1 week | 48.8 | 0.9 | 98.7 | 0.4 | 70.6 | 11.9 | 17.5 | 10.6 | Essentially Free from |
| | | | | | | | | | | Particles |
| Shaking 25° C. | 1 week | 49.0 | 1.0 | 98.7 | 0.4 | 71.8 | 11.8 | 16.4 | 10.3 | Essentially Free from |
| | | | | | | | | | | Particles |
| Freeze/thaw | 5 cycles | 49.0 | 0.9 | 98.7 | 0.3 | 71.2 | 11.8 | 17.1 | 9.7 | Free from Particles |
| 5° C. | 9 months | 49.1 | 1.1 | 98.2 | 0.6 | 69.6 | 12.1 | 18.3 | 10.8 | Free from Particles |
| 25° C. | 9 months | — | 1.7 | 97.5 | 0.9 | 64.5 | 16.4 | 19.1 | 10.2 | Free from Particles |
| 40° C. | 9 months | — | 24.2 | 71.9 | 3.9 | — | — | — | 17.9 | Free from Particles |

| | Storage | Analytical Protein A Chromatography |
|---|---|---|
| Storage condition | Time | Fc Methionine Oxidation (%) |
| — | Initial | 2.2 |
| 5° C. | 9 months | 4.2 |
| 25° C. | 9 months | 4.8 |

F24 is a liquid formulation with the composition 50 mg/mL huMAb P-Selectin, 20 mM histidine acetate, 230 mM sorbitol, 0.02% Poloxamer 188, 10 mM Methionine at pH 5.5

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 49.7 | 0.9 | 98.7 | 0.4 | 71.4 | 11.6 | 17.0 | 9.9 | Free from Particles |
| Shaking 5° C. | 1 week | 49.1 | 0.9 | 98.8 | 0.3 | 70.3 | 9.5 | 17.8 | 9.8 | Essentially Free From |
| | | | | | | | | | | Particles |
| Shaking 25° C. | 1 week | 49.5 | 0.9 | 98.7 | 0.4 | 71.6 | 11.8 | 16.5 | 9.8 | Free From Particles |
| Freeze/thaw | 5 cycles | 48.9 | 0.9 | 98.8 | 0.3 | 71.6 | 11.9 | 16.6 | 9.7 | Free from Particles |
| 5° C. | 9 months | 49.2 | 1.1 | 98.3 | 0.7 | 69.9 | 12.1 | 18.0 | 10.2 | Free from Particles |
| 25° C. | 9 months | — | 1.5 | 97.7 | 0.8 | 65.2 | 16.0 | 18.8 | 10.4 | Free from Particles |
| 40° C. | 9 months | — | 22.0 | 74.6 | 3.4 | — | — | — | 18.3 | Essentially Free from |
| | | | | | | | | | | Particles |

| | Storage | Analytical Protein A Chromatography |
|---|---|---|
| Storage condition | Time | Fc Methionine Oxidation (%) |
| — | Initial | 2.1 |
| 5° C. | 9 months | 4.1 |
| 25° C. | 9 months | 4.2 |

### Example 2

### Preparation of Liquid Formulations for Detailed Formulation Screening

The following huMAb P-selectin liquid formulations F25 to F34 as shown in Table 2 were prepared at a protein concentration ranging from 40 mg/mL to 60 mg/ml:

TABLE 2

| | | Protein | | | |
|---|---|---|---|---|---|
| | | concen- | Poloxamer | | |
| | | tration | 188 concen- | | |
| Code | Buffer | (mg/mL) | tration (%) | Excipient 1 | pH |
| F25 | 20 mM | 40 | 0.01 | 210 mM | 5.0 |
| F26 | Histidine | 60 | 0.01 | Sucrose | 5.0 |
| F27 | Acetate | 40 | 0.03 | | 5.0 |
| F28 | pH 5.5 | 60 | 0.03 | | 5.0 |
| F29 | | 40 | 0.01 | | 6.0 |
| F30 | | 60 | 0.01 | | 6.0 |
| F31 | | 40 | 0.03 | | 6.0 |
| F32 | | 60 | 0.03 | | 6.0 |
| F33 | | 50 | 0.02 | | 5.5 |
| F34 | | 50 | Without | | 5.5 |
| | | | surfactant | | |

huMAb P-Selectin antibody prepared and obtained as described in WO2005/100402 was provided at a concentration of approximately 20 mg/mL in a 20 mM histidine buffer at a pH of approximately 5.5. The huMAb P-selectin antibody used in the examples is a human antibody comprising the heavy chain of SEQ ID NO:24 and the light chain of SEQ ID NO:23.

For the preparation of the liquid formulations huMAb P-Selectin was buffer-exchanged against a diafiltration buffer containing the anticipated buffer composition and concentrated by ultrafiltration to an antibody concentration of approximately 80 mg/mL. After completion of the ultrafiltration operation, the excipients (e.g. trehalose, methionine) were added as stock solutions to the antibody solution. The surfactant was then added as a 125-fold stock solution. Finally the protein concentration was adjusted with a buffer to the final huMAb P-Selectin concentration ranging from approximately 40 mg/mL to 60 mg/mL mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approx. 2.4 mL. These formulations were stored at different climate conditions (5° C., 25° C. and 40° C.) for different intervals of time and stressed by shaking (1 week at a shaking frequency of 200 min-1 at 5° C. and 25° C.) and freeze-thaw stress methods (five cycles at −80° C./+5° C.). The samples were analyzed before and after applying the stress tests as well as after storage by UV spectroscopy, Size Exclusion Chromatography (SEC), Ion exchange chromatography (IEC), Clarity and opalescence of the solution, Analytical Protein A Chromatography and visual inspection as described before in Example 1.

As described before, samples were inspected for the presence of visible particles by using a Seidenader V90-T visual inspection instrument.

| Number of Particles | |
|---|---|
| per Vial | Description |
| 0 | Free from Particles |
| 1-5 | Essentially free from Particles |
| 6-10 | With a few Particles |
| >10 | With many Particles |

### Example 2

### Compositions and Stability Data of Liquid huMAb P-Selectin Drug Product Formulations According to this Invention

F25 is a liquid formulation with the composition 40 mg/mL huMAb P-Selectin, 20 mM histidine acetate, 210 mM sucrose, 0.01% Poloxamer 188, at pH 5.0

| | | | | | | Ion Exchange-HPLC | | | | |
|---|---|---|---|---|---|---|---|---|---|---|
| | | Protein | Size Exclusion-HPLC | | | Main | Acidic | | | |
| | Storage | conc. | HMW | Monomer | LMW | Peak | Species | Basic species | Turbidity | |
| Storage condition | Time | (mg/mL) | (%) | (%) | (%) | (%) | (%) | (%) | (FTU) | Visible particles |
| — | Initial | 40.5 | 0.4 | 99.2 | 0.4 | 72.2 | 9.7 | 14.4 | 6.6 | Free from Particles |
| Shaking 5° C. | 1 week | — | 0.5 | 99.2 | 0.4 | 70.3 | 9.3 | 20.4 | 6.7 | With many Particles |
| Shaking 25° C. | 1 week | 40.5 | 0.5 | 99.1 | 0.4 | 71.4 | 9.0 | 19.7 | 6.6 | Free from Particles |
| Freeze/thaw | 5 cycles | — | 0.5 | 99.2 | 0.4 | 71.3 | 9.3 | 19.4 | 6.5 | Free from Particles |
| 5° C. | 12 months | — | 0.6 | 99.4 | 0.0 | 71.3 | 9.4 | 19.3 | 7.3 | Free from Particles |

| | Storage | Analytical Protein A Chromatography |
|---|---|---|
| Storage condition | Time | Fc Methionine Oxidation (%) |
| — | Initial | 2.0 |
| 5° C. | 12 months | 1.9 |

F26 is a liquid formulation with the composition 60 mg/mL huMAb P-Selectin, 20 mM histidine acetate, 210 mM sucrose, 0.01% Poloxamer 188, at pH 5.0

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 61.1 | 0.5 | 99.2 | 0.4 | 73.0 | 9.6 | 17.7 | 6.9 | Free from Particles |
| Shaking 5° C. | 1 week | — | 0.5 | 99.1 | 0.4 | 71.0 | 9.4 | 19.6 | 8.0 | Essentially free from |
| | | | | | | | | | | Particles |
| Shaking 25° C. | 1 week | 60.4 | 0.5 | 99.1 | 0.4 | 71.5 | 9.2 | 19.4 | 7.5 | Essentially free from |
| | | | | | | | | | | Particles |
| Freeze/thaw | 5 cycles | — | 0.5 | 99.1 | 0.4 | 71.5 | 9.3 | 19.2 | 6.6 | Free from Particles |
| 5° C. | 12 months | — | 0.7 | 99.3 | 0.0 | 72.2 | 9.2 | 18.5 | 8.0 | Free from Particles |

| | Storage | Analytical Protein A Chromatography |
|---|---|---|
| Storage condition | Time | Fc Methionine Oxidation (%) |
| — | Initial | 2.6 |
| 5° C. | 12 months | 2.5 |

F27 is a liquid formulation with the composition 40 mg/mL huMAb P-Selectin, 20 mM histidine acetate, 210 mM sucrose, 0.03% Poloxamer 188, at pH 5.0

| | | | | | | Ion Exchange-HPLC | | | | |
|---|---|---|---|---|---|---|---|---|---|---|
| | | Protein | Size Exclusion-HPLC | | | Main | Acidic | | | |
| | Storage | conc. | HMW | Monomer | LMW | Peak | Species | Basic species | Turbidity | |
| Storage condition | Time | (mg/mL) | (%) | (%) | (%) | (%) | (%) | (%) | (FTU) | Visible particles |
| — | Initial | 40.5 | 0.4 | 99.2 | 0.4 | 71.8 | 9.5 | 18.7 | 6.46 | Free from Particles |
| Shaking 5° C. | 1 week | — | 0.5 | 99.2 | 0.4 | 70.4 | 9.3 | 20.4 | 6.67 | Free from Particles |
| Shaking 25° C. | 1 week | 40.3 | 0.5 | 99.2 | 0.4 | 70.8 | 9.1 | 20.2 | 6.62 | Free from Particles |
| Freeze/thaw | 5 cycles | — | 0.4 | 99.2 | 0.4 | 71.2 | 9.3 | 19.5 | 6.03 | Essentially free from Particles |
| 5° C. | 12 months | — | 0.7 | 99.3 | 0.0 | 72.1 | 9.2 | 18.8 | 7.15 | Free from Particles |

| | Storage | Analytical Protein A Chromatography |
|---|---|---|
| Storage condition | Time | Fc Methionine Oxidation (%) |
| — | Initial | 2.1 |
| 5° C. | 12 months | 2.0 |

F28 is a liquid formulation with the composition 60 mg/mL huMAb P-Selectin, 20 mM histidine acetate, 210 mM sucrose, 0.03% Poloxamer 188, at pH 5.0

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 61.0 | 0.5 | 99.2 | 0.4 | 72.7 | 9.6 | 17.8 | 7.30 | Free from Particles |
| Shaking 5° C. | 1 week | — | 0.5 | 99.1 | 0.4 | 70.9 | 9.3 | 19.8 | 7.13 | Free from Particles |
| Shaking 25° C. | 1 week | 60.5 | 0.5 | 99.1 | 0.4 | 71.0 | 9.2 | 19.8 | 6.82 | Free from Particles |
| Freeze/thaw | 5 cycles | — | 0.5 | 99.1 | 0.4 | 71.4 | 9.3 | 19.3 | 6.95 | Essentially free from |
| | | | | | | | | | | Particles |
| 5° C. | 12 months | — | 0.7 | 99.3 | 0.0 | 72.4 | 9.3 | 18.3 | 7.58 | Free from Particles |

| | Storage | Analytical Protein A Chromatography |
|---|---|---|
| Storage condition | Time | Fc Methionine Oxidation (%) |
| — | Initial | 2.6 |
| 5° C. | 12 months | 2.6 |

F29 is a liquid formulation with the composition 40 mg/mL huMAb P-Selectin, 20 mM histidine acetate, 210 mM sucrose, 0.01% Poloxamer 188, at pH 6.0

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 40.1 | 0.5 | 99.1 | 0.4 | 72.1 | 9.6 | 18.2 | 10.2 | Free from Particles |
| Shaking 5° C. | 1 week | — | 0.6 | 99.1 | 0.4 | 70.7 | 9.6 | 19.7 | 10.9 | Essentially free from |
| | | | | | | | | | | Particles |
| Shaking 25° C. | 1 week | 40.0 | 0.6 | 99.0 | 0.4 | 70.9 | 9.6 | 19.5 | 10.4 | Free from Particles |
| Freeze/thaw | 5 cycles | — | 0.5 | 99.1 | 0.4 | 71.5 | 9.7 | 18.8 | 10.5 | Free from Particles |
| 5° C. | 12 months | — | 0.8 | 99.2 | 0.0 | 72.0 | 10.1 | 17.9 | 11.0 | Free from Particles |

| | Storage | Analytical Protein A Chromatography |
|---|---|---|
| Storage condition | Time | Fc Methionine Oxidation (%) |
| — | Initial | 2.0 |
| 5° C. | 12 months | 2.0 |

F30 is a liquid formulation with the composition 60 mg/mL huMAb P-Selectin, 20 mM histidine acetate, 210 mM sucrose, 0.01% Poloxamer 188, at pH 6.0

| | | | | | | Ion Exchange-HPLC | | | | |
|---|---|---|---|---|---|---|---|---|---|---|
| | | Protein | Size Exclusion-HPLC | | | Main | Acidic | | | |
| | Storage | conc. | HMW | Monomer | LMW | Peak | Species | Basic species | Turbidity | |
| Storage condition | Time | (mg/mL) | (%) | (%) | (%) | (%) | (%) | (%) | (FTU) | Visible particles |
| — | Initial | 59.9 | 0.6 | 99.1 | 0.3 | 72.6 | 9.8 | 17.6 | 12.4 | Free from Particles |
| Shaking 5° C. | 1 week | — | 0.6 | 99.0 | 0.4 | 71.1 | 9.7 | 19.2 | 12.3 | With many Particles |
| Shaking 25° C. | 1 week | 60.1 | 0.7 | 98.9 | 0.4 | 71.4 | 9.7 | 19.0 | 12.3 | Free from Particles |
| Freeze/thaw | 5 cycles | — | 0.6 | 99.0 | 0.4 | 70.8 | 9.6 | 19.6 | 12.3 | Free from Particles |
| 5° C. | 12 months | — | 0.9 | 99.1 | 0.0 | 71.8 | 10.3 | 17.9 | 13.1 | Free from Particles |

| | Storage | Analytical Protein A Chromatography |
|---|---|---|
| Storage condition | Time | Fc Methionine Oxidation (%) |
| — | Initial | 2.0 |
| 5° C. | 12 months | 2.0 |

F31 is a liquid formulation with the composition 40 mg/mL huMAb P-Selectin, 20 mM histidine acetate, 210 mM sucrose, 0.03% Poloxamer 188, at pH 6.0

| | | | | | | Ion Exchange-HPLC | | | | |
|---|---|---|---|---|---|---|---|---|---|---|
| | | Protein | Size Exclusion-HPLC | | | Main | Acidic | | | |
| | Storage | conc. | HMW | Monomer | LMW | Peak | Species | Basic species | Turbidity | |
| Storage condition | Time | (mg/mL) | (%) | (%) | (%) | (%) | (%) | (%) | (FTU) | Visible particles |
| — | Initial | 40.1 | 0.6 | 99.1 | 0.4 | 72.0 | 9.6 | 18.4 | 10.8 | Free from Particles |
| Shaking 5° C. | 1 week | — | 0.6 | 99.1 | 0.4 | 71.1 | 9.6 | 19.3 | 10.7 | Essentially free from Particles |
| Shaking 25° C. | 1 week | 40.5 | 0.6 | 99.0 | 0.4 | 71.0 | 9.6 | 19.4 | 10.4 | Free from Particles |
| Freeze/thaw | 5 cycles | — | 0.6 | 99.1 | 0.4 | 70.7 | 9.7 | 19.6 | 9.98 | Essentially free from Particles |
| 5° C. | 12 months | — | 0.8 | 99.2 | 0.0 | 71.4 | 10.2 | 18.4 | 10.9 | Free from Particles |

| | Storage | Analytical Protein A Chromatography |
|---|---|---|
| Storage condition | Time | Fc Methionine Oxidation (%) |
| — | Initial | 2.0 |
| 5° C. | 12 months | 2.0 |

F32 is a liquid formulation with the composition 60 mg/mL huMAb P-Selectin, 20 mM histidine acetate, 210 mM sucrose, 0.03% Poloxamer 188, at pH 6.0

| | | | | | | Ion Exchange-HPLC | | | | |
|---|---|---|---|---|---|---|---|---|---|---|
| | | Protein | Size Exclusion-HPLC | | | Main | Acidic | | | |
| | Storage | conc. | HMW | Monomer | LMW | Peak | Species | Basic species | Turbidity | |
| Storage condition | Time | (mg/mL) | (%) | (%) | (%) | (%) | (%) | (%) | (FTU) | Visible particles |
| — | Initial | 60.3 | 0.6 | 99.1 | 0.3 | 72.8 | 9.7 | 17.5 | 12.8 | Free from Particles |
| Shaking 5° C. | 1 week | — | 0.6 | 99.0 | 0.4 | 71.1 | 9.6 | 19.3 | 12.9 | With many Particles |
| Shaking 25° C. | 1 week | 60.0 | 0.7 | 98.9 | 0.4 | 71.3 | 9.6 | 19.1 | 12.9 | Free from Particles |
| Freeze/thaw | 5 cycles | — | 0.6 | 99.0 | 0.4 | 71.4 | 9.6 | 19.0 | 12.4 | Free from Particles |
| 5° C. | 12 months | — | 0.9 | 99.1 | 0.0 | 72.0 | 10.1 | 17.9 | 13.0 | Free from Particles |

| | Storage | Analytical Protein A Chromatography |
|---|---|---|
| Storage condition | Time | Fc Methionine Oxidation (%) |
| — | Initial | 2.6 |
| 5° C. | 12 months | 2.6 |

F33 is a liquid formulation with the composition 50 mg/mL huMAb P-Selectin, 20 mM histidine acetate, 210 mM sucrose, 0.02% Poloxamer 188, at pH 5.5

| | | | | | | Ion Exchange-HPLC | | | | |
|---|---|---|---|---|---|---|---|---|---|---|
| | | Protein | Size Exclusion-HPLC | | | Main | Acidic | | | |
| Storage | Storage | conc. | HMW | Monomer | LMW | Peak | Species | Basic species | Turbidity | |
| condition | Time | (mg/mL) | (%) | (%) | (%) | (%) | (%) | (%) | (FTU) | Visible particles |
| — | Initial | 50.5 | 0.5 | 99.2 | 0.3 | 72.4 | 9.6 | 18.0 | 9.81 | Free from Particles |
| Shaking 5° C. | 1 week | 50.2 | 0.5 | 99.1 | 0.4 | 71.2 | 9.5 | 19.3 | 9.39 | With Many Particles |
| Shaking 25° C. | 1 week | 50.2 | 0.6 | 99.1 | 0.4 | 71.4 | 9.4 | 19.2 | 9.71 | Free from Particles |
| Freeze/thaw | 5 cycles | 49.9 | 0.5 | 99.1 | 0.4 | 71.5 | 9.5 | 19.0 | 8.38 | Essentially free from |
| | | | | | | | | | | Particles |
| 5° C. | 18 months | — | 0.6 | 99.4 | 0.1 | 72.1 | 9.5 | 18.4 | 10.7 | Free from Particles |

| | Storage | Analytical Protein A Chromatography |
|---|---|---|
| Storage condition | Time | Fc Methionine Oxidation (%) |
| — | Initial | 2.3 |
| 5° C. | 18 months | 3.3 |

F34 is a liquid formulation with the composition 50 mg/mL huMAb P-Selectin, 20 mM histidine acetate, 210 mM sucrose, without Poloxamer 188, at pH 5.5

| | | | | | | Ion Exchange-HPLC | | | | |
|---|---|---|---|---|---|---|---|---|---|---|
| | | Protein | Size Exclusion-HPLC | | | Main | Acidic | Basic | | |
| | Storage | conc. | HMW | Monomer | LMW | Peak | Species | species | Turbidity | |
| Storage condition | Time | (mg/mL) | (%) | (%) | (%) | (%) | (%) | (%) | (FTU) | Visible particles |
| — | Initial | 50.4 | 0.5 | 99.2 | 0.3 | 72.7 | 9.6 | 17.7 | 9.79 | With Many Particles |
| Shaking 5° C. | 1 week | — | 1.0 | 98.7 | 0.3 | 71.6 | 9.4 | 19.0 | 20.2 | With Many Particles |
| Shaking 25° C. | 1 week | 51.5 | 38.9 | 60.7 | 0.2 | 35.1 | 4.0 | 60.9 | not detectable | With Many Particles |
| Freeze/thaw | 5 cycles | — | 0.5 | 99.1 | 0.4 | 71.3 | 9.6 | 19.2 | 26.8 | With Many Particles |
| 5° C. | 12 months | — | 0.7 | 99.2 | 0.0 | 72.4 | 9.6 | 18.1 | 10.2 | Free from Particles |

| | Storage | Analytical Protein A Chromatography |
|---|---|---|
| Storage condition | Time | Fc Methionine Oxidation (%) |
| — | Initial | 2.3 |
| 5° C. | 12 months | 2.3 |

The stability data presented above show that the Poloxamer 188 containing formulations are of good stability in the presence and absence of the stabilizer methionine, whereas Polysorbate formulations are showing good stability data in the presence of the stabilizer methionine only.

1. A stable liquid pharmaceutical formulation comprising:
40 mg/ml to 200 mg/ml of an antibody against P-selectin;
0.01% to 0.1% of a poloxamer;
5 mM to 100 mM of a buffer;
100 mM to 500 mM of at least one stabilizer;
at a pH in the range from 4.5 to 7.0.

2. The pharmaceutical formulation according to claim 1, wherein the concentration of the antibody against P-selectin is in the range of 40 mg/ml to 100 mg/ml, particularly of 50 mg/ml.

3. The pharmaceutical formulation according to claim 1, wherein the poloxamer is Poloxamer 188.

4. The pharmaceutical formulation according to claim 1, wherein the poloxamer is present in a concentration in the range from 0.01% to 0.05%, particularly of 0.02%.

5. The pharmaceutical formulation according to claim 1, wherein the buffer is a histidine buffer, particularly a histidine acetate buffer.

6. The pharmaceutical formulation according to claim 1, wherein the buffer has a concentration in the range of 10 to 30 mM, particularly of 20 mM.

7. The pharmaceutical formulation according to claim 1, wherein the pH of the formulation is in the range of 5.0 to 6.0, particularly at 5.5.

8. The pharmaceutical formulation according to claim 1, wherein at least one stabilizer is selected from sugars or amino acids.

9. The pharmaceutical formulation according to claim 1, wherein at least one stabilizer is present in a concentration in the range from 140 to 250 mM, particularly in the range from 210 to 230 mM.

10. The pharmaceutical formulation according to claim 8 or 9, wherein the stabilizer is selected from the group consisting of trehalose, sucrose, sorbitol and arginine hydrochloride.

11. The pharmaceutical formulation according to claim 1, wherein the antibody against P-selectin is a human or humanized antibody.

12. The pharmaceutical formulation according to claim 1, wherein the antibody against P-selectin comprises a variable region independently selected from the group consisting of
l) the heavy chain variable domain defined by amino acid sequence SEQ ID NO:2 and the light chain variable domain defined by SEQ ID NO:1;
m) the heavy chain variable domain defined by amino acid sequence SEQ ID NO:4 and the light chain variable domain defined by SEQ ID NO:3;
n) the heavy chain variable domain defined by amino acid sequence SEQ ID NO:6 and the light chain variable domain defined by SEQ ID NO:5;
o) the heavy chain variable domain defined by amino acid sequence SEQ ID NO:8 and the light chain variable domain defined by SEQ ID NO:7;
p) the heavy chain variable domain defined by amino acid sequence SEQ ID NO:10 and the light chain variable domain defined by SEQ ID NO:9;
q) the heavy chain variable domain defined by amino acid sequence SEQ ID NO:12 and the light chain variable domain defined by SEQ ID NO:11;
r) the heavy chain variable domain defined by amino acid sequence SEQ ID NO:14 and the light chain variable domain defined by SEQ ID NO:13;
s) the heavy chain variable domain defined by amino acid sequence SEQ ID NO:16 and the light chain variable domain defined by SEQ ID NO:15;
t) the heavy chain variable domain defined by amino acid sequence SEQ ID NO:18 and the light chain variable domain defined by SEQ ID NO:17;
u) the heavy chain variable domain defined by amino acid sequence SEQ ID NO:20 and the light chain variable domain defined by SEQ ID NO:19; and
v) the heavy chain variable domain defined by amino acid sequence SEQ ID NO:22 and the light chain variable domain defined by SEQ ID NO:21.

13. The pharmaceutical formulation according to claim 1, wherein the heavy chain variable domain of the antibody against P-selectin comprises the amino acid sequence SEQ ID NO:4 and the light chain variable domain of the antibody against P-comprises SEQ ID NO:3.

14. The pharmaceutical formulation according to claim 1, wherein the antibody against P-selectin is a human antibody comprising the heavy chain of SEQ ID NO:24 and the light chain of SEQ ID NO:23.

15. The stable liquid pharmaceutical formulation according to claim 1 comprising:
50 mg/ml of a human antibody against P-selectin comprising the heavy chain of SEQ ID NO:24 and the light chain of SEQ ID NO:23; and
(i) 0.02% Poloxamer 188,
230 mM trehalose, and
20 mM histidine acetate buffer at pH 5.5; or
(ii) 0.02% Poloxamer 188,
210 mM sucrose, and
20 mM histidine acetate buffer at pH 5.5; or
(iii) 0.02% Poloxamer 188,
145 mM arginine hydrochloride, and
20 mM histidine acetate buffer at pH 5.5; or
(iv) 0.02% Poloxamer 188,
230 mM sorbitol, and
20 mM histidine acetate buffer at pH 5.5.

16. The stable liquid pharmaceutical formulation according to claim 1 comprising:
(a) 40 mg/ml of a human antibody against P-selectin comprising the heavy chain of SEQ ID NO:24 and the light chain of SEQ ID NO:23,
0.01% Poloxamer 188,
210 mM sucrose, and
20 mM histidine acetate buffer at pH 5.0; or
(b) 60 mg/ml of a human antibody against P-selectin comprising the heavy chain of SEQ ID NO:24 and the light chain of SEQ ID NO:23,
0.01% Poloxamer 188,
210 mM sucrose, and
20 mM histidine acetate buffer at pH 5.0; or
(c) 40 mg/ml of a human antibody against P-selectin comprising the heavy chain of SEQ ID NO:24 and the light chain of SEQ ID NO:23,
0.03% Poloxamer 188,
210 mM sucrose, and
20 mM histidine acetate buffer at pH 5.0; or
(d) 60 mg/ml of a human antibody against P-selectin comprising the heavy chain of SEQ ID NO:24 and the light chain of SEQ ID NO:23,
0.03% Poloxamer 188,
210 mM sucrose, and
20 mM histidine acetate buffer at pH 5.0; or
(e) 40 mg/ml of a human antibody against P-selectin comprising the heavy chain of SEQ ID NO:24 and the light chain of SEQ ID NO:23,
0.01% Poloxamer 188,
210 mM sucrose, and
20 mM histidine acetate buffer at pH 6.0; or
(f) 60 mg/ml of a human antibody against P-selectin comprising the heavy chain of SEQ ID NO:24 and the light chain of SEQ ID NO:23,
0.01% Poloxamer 188,
210 mM sucrose, and
20 mM histidine acetate buffer at pH 6.0; or
(g) 40 mg/ml of a human antibody against P-selectin comprising the heavy chain of SEQ ID NO:24 and the light chain of SEQ ID NO:23,
0.03% Poloxamer 188,
210 mM sucrose, and
20 mM histidine acetate buffer at pH 6.0; or
(h) 60 mg/ml of a human antibody against P-selectin comprising the heavy chain of SEQ ID NO:24 and the light chain of SEQ ID NO:23,
0.03% Poloxamer 188,
210 mM sucrose, and
20 mM histidine acetate buffer at pH 6.0; or
(i) 50 mg/ml of a human antibody against P-selectin comprising the heavy chain of SEQ ID NO:24 and the light chain of SEQ ID NO:23,
0.02% Poloxamer 188,
210 mM sucrose, and
20 mM histidine acetate buffer at pH 5.5.

20. A method for the prevention or treatment of inflammatory and thrombotic diseases as well as vascular disease pathologies driven by inflammatory and/or thrombotic processes, particularly for use in the prevention or treatment of vascular diseases with underlying atherothrombosis and vascular narrowing such as coronary artery disease (CAD), acute coronary syndrome (ACS), peripheral arterial disease (PAD), peripheral arterial occlusive disease (PAOD), critical limb ischemia (CLI), stenosis after coronary artery bypass grafting (CABG), coronary vein graft disease, restenosis after percutaneous coronary intervention (PCI), dialysis shunt stenosis, dialysis shunt occlusion as well as arterial and deep venous thrombosis and thrombotic thrombocytopenic purpura (TPP), the prevention and treatment of post-ischemic tissue damage caused by myocardial infarction, cerebral ischemic event (e.g. stroke), renal infarction, acute kidney injury, organ transplant rejection and acute leukocyte-mediated lung-injury, the treatment of septic shock, allergic conditions, asthma, chronic obstructive pulmonary disease (COPD), transplant vasculopathy, acute pancreatitis, inflammatory bowel disease, rheumatoid arthritis, atopic dermatitis, psoriasis, sickle cell disease and prevention of tumor metastasis which method comprises administering the stable liquid pharmaceutical formulation according to claim 1, 15 or 16.


SEQUENCE LISTING (24 sequences)

SEQ ID NO: 1 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRNNWPLTFGGGTKVEIK

SEQ ID NO: 2 - protein, 124 aa, Homo sapiens
EVQLVESGGGLVQPGGSLRLSCAASGFTFSSYDMHWVRQATGKGLEWVSGITTAGDTYYPGSVKGRFTISRENAKNSLYLQMNSLRAGDTAVYYCARGRISMDRGVKNNWFDPWGQGTLVTVSS

SEQ ID NO: 3 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 4 - protein, 124 aa, Homo sapiens
EVQLVESGGGLVRPGGSLRLSCAASGFTFSNYDMHWVRQATGKGLEWVSAITAAGDIYYPGSVKGRFTISRENAKNSLYLQMNSLRAGDTAVYYCARGRYSGSGSYYNDWFDPWGQGTLVTVSS

SEQ ID NO: 5 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYHSYPLTFGGGTKVEIK

SEQ ID NO: 6 - protein, 128 aa, Homo sapiens
QVQLVESGGGVVQPGRSLRLSCAASGFTFSSYGMHWVRQAPGEGLEWVAVIWYDGTFKYYADSVKGRFTISRDNSKNTLSLLMNSLRAEDTAVYYCTRGGYYGSGSSFDYYYYGMDVWGQGTTVTVSS

SEQ ID NO: 7 - protein, 107 aa, Homo sapiens; identical to SEQ ID NO: 5

SEQ ID NO: 8 - protein, 117 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDDLDYFDYWGQGTQVTVSS

SEQ ID NO: 9 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIK

SEQ ID NO: 10 - protein, 117 aa, Homo sapiens; identical to SEQ ID NO: 8

SEQ ID NO: 11 - protein, 108 aa, Homo sapiens; identical to SEQ ID NO: 9

SEQ ID NO: 12 - protein, 128 aa, Homo sapiens; identical to SEQ ID NO: 6

SEQ ID NO: 13 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 14 - protein, 124 aa, Homo sapiens
EVQLVESGGGLVQPGGSLRLSCAASGFTFSSYDMHWVRQATGKGLEWVSAISTAGDTYYPGSVKGRFTISRENAKNSLYLQMNSLRVGDTAVYYCARGRFDGSGSYYNDWFDPWGQGTLVTVSS

SEQ ID NO: 15 - protein, 107 aa, Homo sapiens
AIQLTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 16 - protein, 124 aa, Homo sapiens; identical to SEQ ID NO: 14

SEQ ID NO: 17 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRYNWPLTFGGGTKVEIK

SEQ ID NO: 18 - protein, 124 aa, Homo sapiens; identical to SEQ ID NO: 14

SEQ ID NO: 19 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 20 - protein, 119 aa, Homo sapiens
QVQLVESGGGVVQPGRSLRLSCAATGFTFSSYGMHWVRQAPGKGLEWVAVIWYDGSKKYYTDSVKGRFTISRDNSKNTLYLQMNSLRAEDTAVYYCARDQNWIDVFDIWGQGTMVTVSS

SEQ ID NO: 21 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 22 - protein, 124 aa, Homo sapiens
QPQLVQSGAEVKKPGASVKVSCKVSGNTLTELSMHWVRQAPGKGLEWMGGFDPENGEAIYAQKFQGRVTMTADTSTDTAYMDLSSLRSEDTAVYYCATDLAGGSDFYYYGLDVWGQGTTVTVSS

SEQ ID NO: 23 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 451 aa, Homo sapiens
EVQLVESGGGLVRPGGSLRLSCAASGFTFSNYDMHWVRQATGKGLEWVSAITAAGDIYYPGSVKGRFTISRENAKNSLYLQMNSLRAGDTAVYYCARGRYSGSGSYYNDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK